(19) United States
(12) Patent Application Publication
(10) Pub. No.: US 2016/0046684 A1
(43) Pub. Date: Feb. 18, 2016

(54) Methods and compositions for treatment of PTP1B-related diseases

(71) Applicant: BROWN UNIVERSITY, Providence, RI (US)

(72) Inventors: Wolfgang Peti, Barrington, RI (US); Rebecca Page, Barrington, RI (US)

(73) Assignee: BROWN UNIVERSITY, Providence, RI (US)

(21) Appl. No.: 14/826,686

(22) Filed: Aug. 14, 2015

Related U.S. Application Data
(60) Provisional application No. 62/038,511, filed on Aug. 18, 2014.

(51) Int. Cl. C07K 14/47; G01N 33/573

(52) U.S. Cl. CPC C07K 14/4703; G01N 33/573; A61K 38/00

(57) ABSTRACT

Cancer, obesity, and diabetes are examples of PTP1B-related diseases. The invention herein provides embodiments of therapeutic methods and compositions for treating PTP1B related diseases by engineering peptides that bind to the functional spine of PTP1B to alter the conformation of the protein and inhibit a function of PTP1B. Molecules bind to amino acid residues of the functional spine of PTP1B, regulate the catalytic cycle, and treat PTP1B-related diseases. For example, molecules bind to at least one of Tyr152, Tyr153, His175, Thr178, Pro185, Phe191, Leu192, Asn193, Cys215, Gly218, Phe280, Val287, Trp291, Lys292, Leu294, Ser295, and Glu297 to reduce at least one symptom of a PTP1B related disease.

### RELATED APPLICATIONS

This application claims the benefit of U.S. provisional application 62/038,511 filed Aug. 18, 2014 in the U.S. Patent and Trademark Office, which is hereby incorporated by reference in its entirety.

### GOVERNMENT SUPPORT CLAUSE

This invention was made with government support under grant numbers CA53840, GM55989, CA45508, GM100910, GM098482, and S10-RR017269 awarded by the National Institutes of Health. The government has certain rights in the invention.

### TECHNICAL BACKGROUND

Embodiments of the invention contains therapeutic methods and compositions for regulating the catalytic cycle of phosphotyrosine phosphatase to treat PTP1B-related disease by engineering peptides that bind PTP1B and change conformation of the protein to inhibit at least one function of PTP1B.

### BACKGROUND

Phosphotyrosine phosphatase PTP1B is a therapeutic target for potential therapeutic agents to treat diabetes, obesity, and breast tumorigenesis. See, Tonks, N. K. et al. Journal of Biological Chemistry 263:6722-6730 (1988). Efforts to develop therapeutic inhibitors of PTP1B have been frustrated by the chemical properties of the active site. Disruption of the normal patterns of protein phosphorylation results in aberrant regulation of signal transduction and has been implicated in the etiology of a variety of major human diseases. The ability to selectively modulate signaling pathways has therapeutic potential. The first drugs directed against protein tyrosine kinases (PTKs) represented breakthroughs in cancer therapy. For example, the humanized antibody Herceptin® (trastuzumab) targets PTK HER2 (ERBB2), which is amplified and overexpressed in about 25% of breast tumors and is associated with poor prognosis. See, Slamon et al., Science 244:707-712 (1989); Tiwari et al., Anticancer Research 12:419-425 (1992). Treatment with Herceptin® has a low overall success rate, and patients develop resistance to the therapy. Similar problems have limited the success of other PTK-based therapies. See, Engelman et al., Current Opinion in Genetics & Development 18:73-79; 2008; Rexer et al., Critical Reviews in Oncogenesis 17:1-16 (2012). The aforementioned problems remain a major problem for identifying of such alternative therapies.

Focus on PTKs for drug development ignores other major components of phosphorylation-dependent signaling regulation. Protein phosphorylation is reversible and coordinated. Competing activities of kinases and phosphatases are important for determining signaling outcome. The protein tyrosine phosphatases (PTPs), which work in combination with the PTKs, remain a largely untapped resource for drug development.

PTP1B plays a role in down-regulating signaling in response to insulin and leptin plays. See, Tonks, FEBS Journal 280:346-378 (2013). Gene targeting studies demonstrate that PTP1B-null mice are healthy, display enhanced insulin sensitivity, do not develop type 2 diabetes, and are resistant to obesity when fed a high fat diet. See, Elchebly et al., Science 283, 1544-1548 (1999); Klaman et al., Molecular and Cellular Biology 20:5479-5489 (2000). Furthermore, depletion of PTP1B expression with antisense oligonucleotides elicits anti-diabetic and anti-obesity phenotypes in rodents and human subjects. See, Zinker et al., Proc Natl Acad Sci USA 99:11357-11362 (2002). Mice expressing activated alleles of HER2 in mammary glands develop multiple mammary tumors and frequent metastasis to the lung; however, when such mice were crossed with PTP1B-null mice, tumor development was delayed and the incidence of lung metastases was decreased. Conversely, targeted overexpression of PTP1B drove mammary tumorigenesis. See, Lantz et al., Obesity (Silver Spring) 18, 1516-1523 (2010). These observations are evidence that PTP1B plays a role in attenuating insulin signaling and promoting signaling events associated with breast tumorigenesis.

The pharmaceutical industry has mounted major programs to develop small molecule inhibitors that target the active site of PTP1B to treat a PTP1B-related disease. These efforts have been frustrated by technical challenges arising from the chemistry of PTP catalysis. Potent, specific, and reversible inhibitors of PTP1B have been generated; however, the molecules obtained were highly charged thus having limited drug development potential. See, Andersen et al., Topics in Current Genetics 5:201-230 (2004).

There is a need for therapeutic agents to regulate the catalytic site to inhibit of PTP1B.

### SUMMARY

In certain embodiments, a method of treating a subject for a PTP1B related disease includes the steps of administering to a subject a composition that binds to at least one portion of PTP protein distal to an catalytic site to change the conformation of enzymatic PTP protein; inhibiting at least one function of PTP1B compared to the PTP1B function prior to the administering; and decreasing a symptom of the PTP1B related disease to treat the subject for the disease. In one embodiment, the catalytic site is an active site. In one embodiment, the portion of the PTP1B protein bound by the composition is at least one of an α7 helix, an α3 helix, an α3 helix, an L11 loop, an E loop, and an WPD loop of the PTP1B. In one embodiment, the α7 domain of PTP includes glycine at amino acid position 277, phenylalanine at amino acid position 280, valine at amino acid position 287, lysine at amino acid position 292, leucine at amino acid position 294, and serine at amino acid position 295. In one embodiment, the PTP1B has an amino acid sequence including SEQ ID NO: 1, SEQ ID NO: 2, SEQ ID NO: 3, or conservative substitutions thereof. In one embodiment, the portion of PTP1B is a functional spine including at least one amino acid residue selected from the group consisting of: Tyr152, Tyr153, Thr178, Pro185, Asn193, Trp291, Ser295, and Glu297. In another embodiment, the changing step further includes stabilizing the enzyme in an inactive form. In one embodiment, the PTP1B related disease is at least one disease selected from the group consisting of cancer, obesity, and diabetes. In one embodiment, the changing step further includes allosterically modulating catalytic activity of PTP1B. In one aspect, the inhibiting step is performed indirectly. In one embodiment, a WPD loop of the PTP is in an open conformation during the inhibiting step.

In certain embodiments, a composition for treating a PTP1B related disease includes a molecule that binds to a portion of human PTP1B protein distal to a catalytic site thereby changing the conformation of PTP1B, such that at least one function of the PTP protein is inhibited. In one embodiment, the molecule binds to at least one amino acid residue selected from the group consisting of Tyr152, Tyr153, His175, Thr178, Pro185, Phe191, Leu192, Asn193, Cys215, Gly218, Phe280, Val287, Trp291, Lys292, Leu294, Ser295, and Glu297. In one aspect, the molecule is a noncompetitive inhibitor. In one aspect, the molecule is a competitive inhibitor. In one embodiment, the PTP protein has an amino acid sequence including SEQ ID NO: 1, SEQ ID NO: 2, SEQ ID NO: 3, or conservative substitutions thereof. In one embodiment, the molecule has an amino acid sequence including at least one amino acid sequence selected from the group of SEQ ID NO: 1, SEQ ID NO: 2, SEQ ID NO: 3, and conservative substitutions thereof. In one embodiment, changing conformation includes stabilizing PTP1B in an inactive state. In one embodiment, the molecule binds to at least one amino acid residue selected from the group consisting of a tyrosine in a L11 loop, a histidine between the L11 loop and a WPD loop, a threonine in the WPD loop, a proline in an α3 helix, a phenylalanine in the α3 helix, a leucine in the α3 helix, an asparagine in the α3 helix, a cysteine in a PTP loop, a glycine in a PTP loop, a phenylalanine in an α6 loop, a valine in the α6 loop, a tryptophan in an α7 helix, a lysine in the α7 helix, a leucine in the α7 helix, a serine in the α7 helix, and a glutamic acid in the α7 helix.

In certain embodiments, a method of identifying a peptide that inhibits PTP1B includes contacting a mixture of purified PTP1B with a peptide library containing at least one peptide having an amino acid sequence selected from the group consisting of: SEQ ID NO: 1, SEQ ID NO: 2, SEQ ID NO: 3, and conservative substitutions thereof; isolating at least one peptide that bind PTP and alter conformation thereby inhibiting PTP1B; and determining and aligning amino acid sequences of the peptides that bind PTP1B and alter conformation to identify amino acid positions with conserved amino acids. In one embodiment, the isolating step further includes isolating the peptide that restricts movement of amino acid residues Phe191 or Pro185, spacing of α3 helix, or interferes with connection of the WPD loop with the E-loop. In one embodiment, the isolating step includes isolating the peptide that bind PTP1B distal to the active site. In one embodiment, the isolating step further includes isolating peptides that regulate the catalytic cycle of PTP1B.

### BRIEF DESCRIPTION OF THE DRAWINGS

FIG. 1 is a ribbon model of a cys-based protein tyrosine phosphatase in the apo state based on x-ray crystallography results. The inset is a magnification at the conformation of the Q loop, SBL loop, PTP loop, WPD loop, and E loop.

FIG. 2 is a graph of the 2D [¹H,¹⁵N] transverse relaxation optimized spectroscopy (TROSY) nuclear magnetic resonance (NMR) spectrum of PTP1B₁₋₃₀₁.

FIGS. 3A and 3B are overlays of nuclear relaxation time measured at 500 MHz and 850 MHz.

FIG. 3A is an overlay of the R₂ (1/s) values for PTP1B₁₋₃₀₁ measured at 500 MHz and 850 MHz.

FIG. 3A is an overlay of the R₂ (1/s) values for PTP1B₁₋₃₀₁ measured at 500 MHz and 850 MHz.

FIG. 4 is a graph of [¹H, ¹⁵N]het-NOE NMR spectrometry data of PTP1B₁₋₃₀₁ FIG. 5 is a ribbon model of PTP1B₁₋₃₀₁ bound with allosteric inhibitor PDB 1T49 and rotated 90° based on x-ray crystallography results. The active site/PTP loop is shown in cyan, and the WPD loop is shown in red.

FIG. 6 is a ribbon model of amino acid residues of 5-282 of PTP1B bound with active site inhibitor PDB:1C88 based on x-ray crystallography results. The active site/PTP loop is shown in cyan, and the WPD loop is shown in red.

FIGS. 7A and 7B are a graph of chemical shifts (CS) for PTP1B₁₋₃₀₁ bound to TCS-401 and a ribbon model of PTP1B₁₋₃₀₁ bound to TCS-401 based on x-ray crystallography results.

FIG. 7A is a graph of CS for PTP1B₁₋₃₀₁ bound to TCS-401.

FIG. 7B is a ribbon model of PTP1B₁₋₃₀₁ bound to TCS-401 highlighting in red the amino acid residues with CS greater than the mean plus 2σ.

FIGS. 8A and 8B are overlays of NMR nuclear relaxation time data of PTP1B₁₋₃₀₁ bound to TCS-401 and apo PTP1B₁₋₃₀₁.

FIG. 8A is an overlay of the R₂ (1/s) values for PTP1B₁₋₃₀₁ bound to TCS-401 and apo PTP1B₁₋₃₀₁.

FIG. 8B is an overlay of the R₁ (1/s) values for PTP1B₁₋₃₀₁ bound to TCS-401 and apo PTP1B.

FIG. 9 is an overlay of ¹⁵N-hetNOE data of PTP1B₁₋₃₀₁ bound to TCS-401 and apo PTP1B₁₋₃₀₁.

FIGS. 10A and 10B are a graph of CS of PTP1B₁₋₃₀₁ bound to allosteric inhibitor PDB 1T4J and a ribbon model of PTP1B₁₋₃₀₁ bound to an allosteric inhibitor.

FIG. 10A a graph of CS of PTP1B₁₋₃₀₁ bound to allosteric inhibitor PDB 1T4J.

FIG. 10B is a ribbon model of PTP1B₁₋₃₀₁ bound to allosteric inhibitor PDB 1T4J based on x-ray crystallography results.

FIGS. 11A and 11B are overlays of NMR nuclear relaxation time data of PTP1B₁₋₃₀₁ bound to allosteric site inhibitor 3-(3,5-Dibromo-4-hydroxy-benzoyl)-2-ethyl-benzofuran-6-sulfonic acid-(4-(thiazol-2-ylsulfamyl)-phenyl)-amide (CAS number 765317-72-4) and apo PTP and a graph of 15N-hetNOE results for titration with an allosteric inhibitor of PTP1B.

FIG. 11A is an overlay of R₂ (1/s) values for apo PTP1B₁₋₃₀₁ and PTP1B₁₋₃₀₁ bound to the allosteric inhibitor.

FIG. 11B is an overlay of R₁ (1/s) values for apo PTP1B₁₋₃₀₁ and PTP1B₁₋₃₀₁ bound to the allosteric inhibitor.

FIG. 12 is an overlay of ¹⁵N-hetNOE data comparing the dynamics of PTP1B₁₋₃₀₁ bound to allosteric inhibitor 3-(3,5-Dibromo-4-hydroxy-benzoyl)-2-ethyl-benzofuran-6-sulfonic acid-(4-(thiazol-2-ylsulfamyl)-phenyl)-amide (CAS number 765317-72-4) and apo PTP1B₁₋₃₀₁.

FIGS. 13A and 13B are ribbon models of ligand-free PTP1B₁₋₃₀₁ and ligand-bound PTP1B₁₋₃₀₁ based on x-ray crystallography results.

FIG. 13A is a ribbon model of PTP1B is an open conformation. The PTP loop/active site and WPD loop are dark gray.

FIG. 13B is a ribbon model of PTP is a closed conformation. The PTP loop/active site and WPD loop are dark gray.

FIGS. 14A and 14B are ribbon models of PTP1B₁₋₃₀₁ with the α3 helix, the α7 helix, and D181 labeled based on x-ray crystallography analysis.

FIG. 14A is a ribbon model of PTP1B₁₋₃₀₁ with the α3 helix, the α7 helix, and D181 labeled.

FIG. 14B is a ribbon model of PTP1B₁₋₃₀₁ bound to TCS-401 with the α3 helix, the α7 helix, and D181 labeled.

FIG. 15 is a graph of results of enzyme assays comparing the activities of wild-type PTP1B₁₋₃₀₁ to mutant PTP1B_{P185G}.

FIGS. 16A and 16B are ribbon models of PTP1B₁₋₃₀₁ in open and closed conformations.

FIG. 16A is a ribbon model of PTP1B₁₋₃₀₁ in an open conformation.

FIG. 16B is a ribbon model of PTP1B₁₋₃₀₁ in a closed conformation.

FIG. 17 is a graph of enzyme assay data of the of wild-type PTP1B₁₋₃₀₁ and mutants: PTP1B_{Y152A/F}, PTP1B_{Y153A/F}, and PTP1B_{YAYA}.

FIGS. 18A and 18B are overlays of NMR nuclear relaxation time data of wild-type PTP1B₁₋₃₀₁ and PTP1B_{YAYA}.

FIG. 18A is an overlay of R₁ (1/s) values for wild-type PTP1B₁₋₃₀₁ and PTP1B_{YAYA}.

FIG. 18B is an overlay of R₂ (1/s) values for wild-type PTP1B₁₋₃₀₁ and PTP1B_{YAYA}.

FIGS. 19A and 19B are a graph of CS of wild-type PTP1B₁₋₃₀₁ bound to TCS-401 and a ribbon model of PTP1B₁₋₃₀₁ bound to TCS-401 based on x-ray crystallography results.

FIG. 19A a graph of CS of wild-type PTP1B₁₋₃₀₁ bound to TCS-401.

FIG. 19B is a ribbon model of wild-type PTP1B₁₋₃₀₁ bound to TCS-401 with amino acid residues highlighted in red that had a CS greater than the mean plus 2σ.

FIGS. 20A and 20B are overlays of NMR nuclear relaxation time data comparing the dynamics of wild-type PTP1B bound to TCS-401 and PTP1B_{YAYA} bound to TCS-401.

FIG. 20A is an overlay of R₁ (1/s) values for wild-type PTP1B₁₋₃₀₁ bound to TCS-401 and PTP1B_{YAYA} bound to TCS-401.

FIG. 20B is an overlay of R₂ (1/s) values for wild-type PTP1B₁₋₃₀₁ bound to TCS-401 and PTP bound to TCS-401.

FIG. 21 is a photograph of a gel of the effects of insulin signaling for each of a control, wild-type PTP1B, and PTP1B_{YAYA}.

FIGS. 22A-22D are ribbon models of the spine of PTP1B, wild-type PTP1B, wild-type PTP1B bound to TCS-401, PTP1B_{YAYA}, and PTP1B_{YAYA} bound to TCS-401 based on x-ray crystallography data. The PTP/active site loop is shown in marine, the WPD loop is shown in magenta, and the α7 helix is shown in orange.

FIG. 22A is a ribbon model of the dynamic spine of PTP1B.

FIG. 22B is a ribbon model comparing the structures of wild-type PTP1B (gray) to PTP1B_{YAYA} (teal).

FIG. 22C is a ribbon model comparing the structures of wild-type PTP1B (gray) bound to TCS-401 to PTP1B_{YAYA} bound to TCS-401 (green).

FIG. 22D is a ribbon model comparing the structures of PTP1B_{YAYA} (teal) to PTP1B_{YAYA} bound to TCS-401 (green).

FIGS. 23A and 23B is an overlay 2D [¹H,¹⁵N] TROSY NMR spectra and an overlay of CS of PTP1B₁₋₃₀₁ and PTP1B_{YAYA}.

FIG. 23A is a graph of CS of PTP1B₁₋₃₀₁ and PTP1B_{YAYA}.

FIG. 23B is an overlay 2D [¹H,¹⁵N] TROSY NMR spectra of PTP1B₁₋₃₀₁ (red) and PTP1B_{YAYA} (blue).

FIG. 24 is a graph of phosphatase activity of PTP1B₁₋₃₀₁, PTP1B_{Δ7}, and PTP1B_{YAYA}.

FIG. 25 is a graph of melting temperatures of PTP1B₁₋₃₀₁, PIT 1%₇, and PTP1B_{YAYA} measured by circular dichroism (CD) spectropolarimetry.

FIGS. 26A and 26B are a ribbon model of PTP1B_{YAYA} and a drawing representing domain interactions in PTP1B. The active site/PTP loop is shown as cyan, and the WPD loop is shown as red.

FIG. 26A is a ribbon model of PTP1B_{YAYA} having α7 helix, N193, and the locations of the YAYA mutation labeled.

FIG. 26B is a drawing representing interactions among L11 loop, α7 helix, α3 helix, and WPD for regulation of PTP1B activity.

FIG. 27 is a graph of enzyme activity assays of wild-type PTP1B₁₋₃₀₁ and mutants with point mutations in the α3 helix.

FIGS. 28A and 28B are overlays of CS of wild-type PTP1B₁₋₃₀₁ and PTP1B_{N193A} and of 2D [¹H, ¹⁵N] TROSY NMR spectra of PTP1B₁₋₃₀₁ and PTP1B_{N193A}.

FIG. 28A is an overlay of CS of wild-type PTP1B₁₋₃₀₁ and PTP1B_{N193A}.

FIG. 28B is an overlay of 2D [¹H, ¹⁵N] TROSY NMR spectra of PTP1B₁₋₃₀₁ (red) and PTP1B_{N193A} (blue).

FIGS. 29A and 29B are a graph of CS for PTP1B_{N193A} bound to TCS-401 and a ribbon model of PTP1B₁₋₃₀₁ bound to TCS-401 based on x-ray crystallography results.

FIG. 29A is a graph of CS for PTP1B_{N193A} bound to TCS-401 based on x-ray crystallography results.

FIG. 29B is a ribbon model of PTP1B_{N193A} bound to TCS-401 highlighting in red the amino acid residues with a CS greater than the mean plus 2σ.

FIGS. 30A-30D are overlays of NMR nuclear relaxation time data of wild-type PTP1B bound to TCS-401 and PTP1B_{N193A} bound to TCS-401.

FIG. 30A is an overlay of R₁ (1/s) values for wild-type PTP1B₁₋₃₀₁ bound to TCS-401 and PTP1B_{N193A} bound to TCS-401.

FIG. 30B is an overlay of R₂ (1/s) values for wild-type PTP1B₁₋₃₀₁ bound to TCS-401 and PTP1B_{N193A} bound to TCS-401.

FIG. 30C is an overlay of R₁ (1/s) values for wild-type PTP1B₁₋₃₀₁ and PTP1B_{N193A}.

FIG. 30D is an overlay of R₂ (1/s) values for wild-type PTP1B₁₋₃₀₁ and PTP1B_{N193A}.

FIGS. 31A-31C are ribbon models comparing the conformations of wild-type PTP1B₁₋₃₀₁, PTP1B₁₋₃₀₁ bound to TCS-401, PTP1B_{N193A} bound to TCS-401, and PTP1B_{N193A} based on x-ray crystallography data.

FIG. 31A is a ribbon model comparing the structures of wild-type PTP1B (gray) to PTP1B_{N193A} (brown).

FIG. 31B is a ribbon model comparing the structures of wild-type PTP1B bound to TCS-401 (gray) to PTP1B_{N193A} bound to TCS-401 (yellow).

FIG. 31C is a ribbon model comparing the structures of PTP1B_{N193A} (brown) to PTP1B_{N193A} bound to TCS-401 (yellow).

FIGS. 32A and 32B are a graph of enzyme activity assays of wild-type PTP1B₁₋₃₀₁ and mutants with mutations in the α7 helix.

FIG. 32A is a graph of enzyme assays comparing the activities of wild-type PTP1B₁₋₃₀₁ to mutants with point mutations in the α7 helix.

FIG. 32B is a graph of enzyme assays comparing the activities of wild-type PTP1B₁₋₃₀₁ to PTP1B_{1-284/Δ7}.

FIGS. 33A and 33B is a graph of CS of PTP1B₁₋₂₈₄ and an overlay of 2D [¹H, ¹⁵N] TROSY NMR spectra of wild-type PTP1B₁₋₃₀₁ and PTP1B₁₋₂₈₄.

FIG. 33A is a graph of CS for PTP1B₁₋₂₈₄.

FIG. 33B is an overlay of 2D [¹H, ¹⁵N] TROSY NMR spectra of wild-type PTP1B₁₋₃₀₁ and PTP1B₁₋₂₈₄.

FIGS. 34A and 34B are overlays of NMR nuclear relaxation time data comparing the dynamics of wild-type PTP1B₁₋₃₀₁ (gray) to PTP1B₁₋₂₈₄ (black).

FIG. 34A is an overlay of R₁ (1/s) values for wild-type PTP1B₁₋₃₀₁ and PTP1B₁₋₂₈₄.

FIG. 34B is an overlay of R₂ (1/s) values for wild-type PTP1B₁₋₃₀₁ and PTP1B₁₋₂₈₄.

FIGS. 35A and 35B are a graph of CS for PTP1B_{Δ7} bound to TCS-401 and a ribbon model of PTP1B_{Δ7} bound to TCS-401 based on x-ray crystallography results.

FIG. 35A is a graph of CS for PTP1B_{Δ7} bound to TCS-401.

FIG. 35B is a ribbon model of PTP1B_{Δ7} bound to TCS-401 highlighting in red the amino acid residues with a CS greater than the mean plus 2σ.

FIGS. 36A and 36B are overlays of NMR nuclear relaxation time data of wild-type PTP1B₁₋₃₀₁ bound to TCS-401 (gray) to PTP1B_{Δ7} bound to TCS-401 (black).

FIG. 36A is an overlay of R₁ (1/s) values for wild-type PTP1B₁₋₃₀₁ bound to TCS-401 (gray) to PTP1B_{Δ7} bound to TCS-401 (black).

FIG. 36B is an overlay of R₂ (1/s) values for wild-type PTP1B₁₋₃₀₁ bound to TCS-401 (gray) to PTP1B_{Δ7} bound to TCS-401 (black).

FIGS. 37A-37C are ribbon models comparing the conformations of wild-type PTP1B, wild-type PTP1B bound to TCS-401, PTP1B₁₋₂₈₄ bound to TCS-401, and PTP1B₁₋₂₈₄ based on x-ray crystallography data. The PTP loop/active site is shown in marine, and the WPD loop is shown in magenta.

FIG. 37A is a ribbon model comparing the structures of wild-type PTP1B bound to TCS-401 (gray) to PTP1B₁₋₂₈₄ bound to TCS-401 (purple).

FIG. 37B is a ribbon model comparing the structures of PTP1B₁₋₂₈₄ bound to TCS-401 (purple) to PTP1B₁₋₂₈₄ (cyan).

FIG. 37C is a ribbon model comparing the structures of PTP1B_{N193A} (brown) to PTP1B_{N193A} bound to TCS-401 (yellow).

FIGS. 38A and 38B are overlays of NMR nuclear relaxation time data comparing the dynamics of apo PTP1B, PTP1B bound to TCS-401, and PTP1B bound to an allosteric inhibitor.

FIG. 38A is an overlay of R₂R₁ versus R₂/R₁ values for apo wild-type PTP1B₁₋₃₀₁ (squares), wild-type PTP1B₁₋₃₀₁ bound to TCS-401 (diamonds), and wild-type PTP1B₁₋₃₀₁ bound to an allosteric inhibitor (circles).

FIG. 38B is an overlay of R₂R₁ versus R₂/R₁ values for apo PTP1B₁₋₂₈₄ (squares), PTP1B₁₋₂₈₄ bound to TCS-401 (diamonds), and PTP1B₁₋₂₈₄ bound to an allosteric inhibitor (circles).

FIGS. 39A and 39B are overlays of NMR nuclear relaxation time data of amino acid residues 175-205 (WPD and α3 helix) and 275-301 (α6 helix and α7 helix) within apo PTP1B, PTP1B bound to TCS-401, and PTP1B bound to an allosteric inhibitor.

FIG. 39A is an overlay of R₂R₁ versus R₂/R₁ values amino acid residues 175-205 (WPD and α3 helix) and 275-301 (α6 helix and α7 helix) within apo wild-type PTP1B₁₋₃₀₁ (squares), wild-type PTP1B₁₋₃₀₁ bound to TCS-401 (diamonds), and wild-type PTP1B₁₋₃₀₁ bound to an allosteric inhibitor (circles).

FIG. 39B is an overlay of R₂R₁ versus R₂/R₁ values for amino acid residues 175-205 (WPD and α3 helix) and 275-284 (α6 helix) within apo PTP1B₁₋₂₈₄ (squares), PTP1B₁₋₂₈₄ bound to TCS-401 (diamonds), and PTP1B₁₋₂₈₄ bound to an allosteric inhibitor (circles).

FIG. 40 is a graph of enzyme assays comparing the activities of wild-type PTP1B₁₋₃₀₁ to mutants with point mutations in the α3 helix.

FIG. 41 is an overlay of 2D [¹H, ¹⁵N] TROSY NMR spectra of wild-type PTP1B₁₋₃₀₁ (black) and PTP1B_{L192A} (gray).

FIG. 42 is an overlay of CS comparing wild-type PTP1B₁₋₃₀₁ with assigned peaks shown in red to PTP1B_{L192A} with assigned peaks shown in green.

FIGS. 43A and 43B are overlays of NMR nuclear relaxation time data of wild-type PTP1B₁₋₃₀₁ (black) and PTP1B_{L192A} (gray).

FIG. 43A is an overlay of R₁ (1/s) values for wild-type PTP1B₁₋₃₀₁ and PTP1B_{L192A}.

FIG. 43B is an overlay of R₂ (1/s) values for wild-type PTP1B₁₋₃₀₁ and PTP1B_{L192A}.

FIGS. 44A and 44B are a graph of CS for PTP1B_{L192A} bound to TCS-401 and a ribbon model of PTP1B_{L192A} bound to TCS-401 based on x-ray crystallography results.

FIG. 44A is a graph of CS for PTP1B_{L192A} bound to TCS-401.

FIG. 44B is a ribbon model of PTP1B_{L192A} bound to TCS-401 highlighting in red the amino acid residues with a CS greater than the mean plus 2σ.

FIGS. 45A and 45B are overlays of NMR nuclear relaxation time data of wild-type PTP1B₁₋₃₀₁ bound to TCS-401 (gray) and PTP1B_{L192A} bound to TCS-401 (black).

FIG. 45A is an overlay of R₁ (1/s) values for wild-type PTP1B₁₋₃₀₁ bound to TCS-401 (gray) and PTP1B_{L192A} bound to TCS-401 (black).

FIG. 45B is an overlay of R₂ (1/s) values for wild-type PTP1B₁₋₃₀₁ bound to TCS-401 (gray) and PTP1B_{L192A} bound to TCS-401 (black).

FIGS. 46A-46C are ribbon models comparing the conformations of wild-type PTP1B, wild-type PTP1B bound to TCS-401, PTP1B_{L192A} bound to TCS-401, and PTP1B_{L192A} based on x-ray crystallography data.

FIG. 46A is a ribbon model comparing the structures of wild-type PTP1B (gray) to PTP1B_{L192A} (violet).

FIG. 46B is a ribbon model comparing the structures of PTP1B_{L192A} bound to TCS-401 (salmon) to wild-type PTP1B₁₋₃₀₁ bound to TCS-401 (gray).

FIG. 46C is a ribbon model comparing the structures of PTP1B_{L192A} (violet) to PTP1B_{L192A} bound to TCS-401 (salmon).

FIG. 47 is a ribbon model of the WPD loop of PTP1B containing two loops in green and pink.

FIGS. 48A-48D are ribbon models comparing conformations of the α3 helix and WPD domain of wild-type PTP1B, wild-type PTP1B bound to TCS-401, PTP1B_{L192A} bound to TCS-401, and PTP1B_{L192A} based on x-ray crystallography data.

FIG. 48A is a ribbon model comparing conformations of the α3 helix and WPD domain within wild-type PTP1B (gray) to PTP1B_{L192A} (purple).

FIG. 48B is a ribbon model comparing conformations of the α3 helix and WPD domain within PTP1B_{L192A} bound to TCS-401 (salmon) to wild-type PTP1B₁₋₃₀₁ bound to TCS-401 (gray).

FIG. 48C is a ribbon model comparing conformations of the α3 helix and WPD domain within PTP1B_{L192A} (purple) to PTP1B_{L192A} bound to TCS-401 (salmon).

FIG. 48D is a ribbon model comparing the structures of PTP1B_{L192A} (purple) to PTP1B_{L192A} bound to TCS-401 (salmon) in relation to R221.

FIG. 49 is a ribbon model comparing conformations of PTP1B_{L192A} bound to TCS-401 (salmon) to wild-type PTP1B₁₋₃₀₁ bound to TCS-401 (gray) with relevant amino acids labeled.

### DETAILED DESCRIPTION

Disruption of protein phosphorylation and signal transduction patterns is associated with a variety of major human diseases, such as diabetes, cancer, and obesity. See, Krishnan et al., Nature Chemical Biology 10:558-566 (2014). For example, PTP1B is overexpressed in breast cancer tumors and promotes tumorigenesis. Ibid. PTP1B is a therapeutic target for diabetes and obesity and plays a positive role in HER2 signaling in breast tumorigenesis. Embodiments of the invention herein are compositions and methods that regulate the catalytic site of PTP1B to treat a PTP1B related disease.

Disruption of the normal patterns of protein phosphorylation results in aberrant regulation of signal transduction. The ability to modulate signaling pathways selectively by simultaneously targeting different signaling enzymes and processes holds enormous therapeutic potential, and is more effective than targeting individual PTKs alone.

PTP1B function is not restricted to metabolic regulation, as it is over-expressed in breast tumors together with HER2. See, Lantz et al., Obesity (Silver Spring), 18, 1516-1523 (2010); Wiesmann et al., Nat. Struct. Mol. Biol., 11, 730-737 (2004). Therefore, inhibition of PTP1B function represents a therapeutic strategy not only to address diabetes and obesity, but also mammary tumorigenesis and malignancy. Consequently new approaches to inhibition of PTP1B, which circumvent the problems with active site-directed small molecule inhibitors, are required to reinvigorate drug development efforts against this highly validated target.

Embodiments of the invention herein contain an inhibitor of PTP1B that binds to sites on the enzyme to regulate the catalytic cycle of the protein.

PTP1B exists in vivo as a longer protein of about 50 kDa serves a regulatory function including direct modulation of activity. See, Metallo, Curr. Opin. Chem. Biol., 14, 481-488 (2010). PTP1B was purified originally from human placenta as a 37 kDa catalytic domain containing amino acid residues 1-321, in which the C-terminal segment has been deleted from the 50 kDa protein. See, Tonks et al., FEBS J., 280, 346-378 (2013). One embodiment of PTP1B₁₋₃₂₁ is provided herein as SEQ ID NO: 1. This portion of PTP1B is stable, readily expressed, and easily purified as a recombinant protein; therefore, it has been the focus of attention to date for mechanistic analysis, as well as for drug screening.

### Pharmaceutical Compositions

In one aspect of the present invention, pharmaceutical compositions are provided, such that these compositions contain a molecule that binds PTP1B distal to a catalytic site, so that the conformation of PTP1B is changed. In certain embodiments, these compositions optionally further contain one or more additional therapeutic agents. In certain embodiments, the additional therapeutic agent or agents are selected from the group consisting of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

As used herein, the phrase “pharmaceutically acceptable carrier” covers solvents, diluents, or other liquid vehicle, dispersion or suspension aids, surface active agents, isotonic agents, thickening or emulsifying agents, preservatives, solid binders, lubricants and the like, as suited to the particular dosage form desired. Remington Twenty-second Edition “The Science and Practice of Pharmacy, Pharmaceutical Press 2012 discloses various carriers used in formulating pharmaceutical compositions and known techniques for the preparation thereof. Examples of materials which serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants may also be present in the composition, according to the judgment of the formulator.

The Examples herein use a variety of biophysical and biochemical strategies to analyze PTP1B to identify amino acids that regulate catalytic activity of PTP1B. Methods of treatment and compositions herein utilize these amino acids as allosteric drug interaction sites. These Examples contain non-limiting embodiments of methods and therapeutic compositions to treat PTP1B related diseases.

### Example 1

### Creating a Homology Model of PTP

A homology model of the C-terminal segment of PTP1B was constructed using Schrodinger's Prime module. A BLAST sequence homology search encompassing amino acid residues (261-414) afforded 52 hits with 22-100% sequence homology in the NCBI PDB (non-redundant). Of the 52, 5 structures were selected for model building based on the following criteria: BLAST bit score, percent identities, percent positives, percent gaps, and x-ray structure resolution. As hits with 100% identity corresponded to PTP1B database sequence entries, they were excluded, with the exception of 1EEN_A for which the structure of amino acid residues 261-282 was determined (α7 helix). The following structures were employed as templates for homology model building: 1EEN_A (1.9 Å), 2GRX_C (3.3 Å), 2KGL_A (NMR), 1RSS_A (1.9 Å), and 3FEO_A (2.5 Å).

Once alignments were performed, comparative model building involved the following steps. Query backbone atoms were superimposed to the aligned template to identify identical side chain atoms. Then, insertions and deletions from the alignment were constructed and removed, respectively. Ab initio methods were used to close gaps using a backbone dihedral library. After completion of the ligation of template junction, minimization of non-conserved amino acid residues and amino acid residues for which no correspondence was found was performed.

The “Build Structure” step used the OPLS_{—}2005 force field, and reference amino acid residue side-chain and dihedral libraries extracted from the PDB for structure prediction. A total of five models were successively generated. For the final model, the 2GRX (TonB, a cytoplasmic membrane protein) template was excluded in favor of 2KGL (MESD, an endoplasmic reticulum-associated chaperone), affording a more complete structure with the least number of discontinuities.

The homology model was ligated to a known PTP1B structure, 1t4j.pdb, that was crystallized at 2.7 Å with an inhibitor bound in the allosteric site, locking the open-form conformation. See, Nat Struct Mol Bio V 11, pp 730-737(2004). Helices α3, α6 and α7 were observed to stabilize the closed-form of PTP1B in the presence of substrate, and allosteric inhibitors were observed to prevent this closure. The R371SR373-containing helix of the C-terminus was observed to be a hinge region during allosteric inhibition. This helix rotated into proximity with helices α6 and α7. The predicted PTP1B structure was employed for docking studies with allosteric inhibitor PDB 1T4J.

### Example 2

### Preparing PTP1B for Dynamics Simulations

PTP1B was prepared for minimization and molecular dynamics simulations as described for docking studies. Preliminary minimization of the PTP1B complex with two molecules of PDB 1 T4J was performed using the steepest descents method with the OPLS 2005 force field and water as solvent over 3000 steps. Molecular dynamics calculations were then performed on the minimized structure. Molecular dynamics were performed at 300K, 1.5 fs, 1 ps equilibration and 50 ps stimulation. The protein backbone was constrained, while the ligands and amino acid residues around the ligands were treated. Final minimization was performed using the Polak-Ribier conjugate gradient method over 500 iterations to a convergence of 0.05 Å RMSD. Final potential and kinetic energy were found to be −369.08 and 10,305.46 kJ/mol, respectively.

Polak-Ribier Conjugate Gradient Minimization

| | | |
|---|---|---|
| Total Energy | −83173.5000 | kJ/mol |
| Stretch | 7086.2427 | kJ/mol |
| Bend | 4231.3643 | kJ/mol |
| Torsion | 5448.0752 | kJ/mol |
| Improper Torsion | 124.0153 | kJ/mol |
| VDW | −2291.7546 | kJ/mol |
| Electrostatic | −71704.9766 | kJ/mol |
| Explicit Hydrogen Bonds | 0.0000 | kJ/mol |
| Cross Terms | 0.0000 | kJ/mol |
| Solvation | −26066.4688 | kJ/mol |

### Example 3

### Creating and Purifying PTP1B Constructs

PTP1B_{cat} (amino acid residues 1-301) was produced as shown in Krishnan et al., Nat. Chem. Biol., 10: 558-566 (2014). In one embodiment, PTP1B₁₋₃₀₁ has an amino acid sequence substantially similar to SEQ ID NO: 2. PTP1B₁₋₂₈₄ (PTP1B_{Δ7}) and mutants of PTP1B_{cat} and PTP1B_{Δ7} were created using site-directed mutagenesis of PTP1B_{cat}. In one embodiment, PTP1B₁₋₂₈₄ (PTP1B_{Δ7}) has an amino acid sequence substantially similar to SEQ ID NO: 3. In one embodiment, mutants PTP1B_{N193A}, PTP1B_{YAYA}, PTP1B_{L192A}, PTP1B_{H175A}, PTP1B_{W291}, and PTP1B_{T178A} contain point mutations of an amino acid sequence substantially similar to SEQ ID NO: 2. In another embodiment, PTP1B_{H175A} contains a point mutation of an amino acid sequence substantially similar to SEQ ID NO: 3. The sequence listing material in computer readable form ASCII text file (8 kilobytes) created Aug. 12, 2015 entitled “02268-098_Sequence_Listing”, containing sequence listings numbers 1-3, has been electronically filed herewith and is incorporated by reference herein in its entirety.

Constructs were expressed in E. coli and purified. Wild-type and mutants of PTP1B were purified by Ni²⁺-affinity chromatography and size exclusion chromatography (SEC, Superdex 75 26/60) using 50 mM HEPES pH 6.8, 150 mM NaCl, 0.5 mM TCEP or 50 mM HEPES pH 7.5, 150 mM NaCl, 0.5 mM TCEP (for PTP1B_{Δ7}) as the final buffer. ²H, ¹⁵N, ¹³C-labeled PTP1B constructs were expressed in E. coli cultures grown in M9 minimal media containing 4 g/L ¹³C-D-glucose, 1 g/L ¹⁵N—NH⁴Cl and 100% D₂O. Cultures were grown at 37° C. and 250 rpm to a final OD600 of about 0.6. Protein expression was induced with the addition of 1 mM IPTG and cultures were incubated for about 20 hours (18° C., 250 rpm). Single ¹⁵N-isotopically labeled amino acid samples (¹⁵N-L-Valine, ¹⁵N-L-Tyrosine, ¹⁵N-L-Phenylalanine or ¹⁵N-L-Leucine) were expressed as previously described. Protein yields were about 46 mg/L in Luria broth, about 34 mg/L in ²H, ¹⁵N M9 medium and about 19 mg/L in ²H, ¹⁵N, ¹³C M9 medium.

To purify the protein, cell pellets were resuspended in ice-cold lysis buffer (50 mM Tris, pH 8.0, 500 mM NaCl, 5 mM imidazole, 0.1% Triton X-100 and EDTA-free protease inhibitor tablets (Roche)) and lysed by high-pressure cell homogenization (Avestin C3 Emulsiflex). The bacterial lysate was clarified by centrifugation at 45,000 g for 60 minutes at 4° C. The supernatant was loaded onto a HisTrap HP column (GE Healthcare) equilibrated with 50 mM Tris, pH 7.5, 5 mM imidazole and 500 mM NaCl, and the His⁶-tagged protein was eluted using an imidazole gradient of 5-500 mM. Fractions containing PTP1B were pooled and cleaved with tobacco etch virus (TEV) protease overnight at 4° C. while being dialyzed against 50 mM Tris, pH 8.0, 500 mM NaCl. Cleaved protein was further purified using Ni²⁺-NTA immobilized metal affinity chromatography followed by size exclusion chromatography (SEC; Superdex 75 26/60; GE Healthcare), equilibrated in NMR buffer (50 mM HEPES, pH 6.8, 150 mM NaCl, 0.5 mM TCEP) to a purity of greater than 98% and had a final yield of 40 mg PTP1B₁₋₃₀₁ or 20 mg PTP1B₁₋₂₈₄ (PTP1B_{Δ7}) and mutants of PTP1B_{cat} and PTP1B_{Δ7} per liter of LB cell culture. Purified PTP1B₁₋₃₀₁, PTP1B₁₋₂₈₄ (PTP1B_{Δ7}), and mutants thereof were concentrated to 0.15 mM, 0.015 mM, or 0.2 mM. For long-term storage, the protein was flash frozen in liquid nitrogen and stored at 80° C.

### Example 4

### Analysis of Apo PTP1B₁₋₃₀₁

For crystallization and structure determination, PTP1B_{cat}, PTP1B₆₃ and mutants of PTP1B_{cat} and PTP1B_{Δ7} were purified as previously described with the exception that the final protein buffer was 20 mM Tris pH 7.5, 25 mM NaCl, 0.2 mM EDTA, 0.5 mM TCEP. Crystals were obtained using sitting drop vapor diffusion in a variety of conditions including 0.1 M Tris, pH 7.4, 20% PEG8000, 0.2 M MgCl₂. Crystals were cryo-protected by a 10 second soak in mother liquor supplemented with 30% glycerol and immediately flash frozen in liquid nitrogen. X-ray data were collected on a single crystal at 112 K using a Rigaku FR-E+ Superbright rotating copper anode X-ray generator with a Saturn 944 HG CCD detector (Brown University Structural Biology Facility).

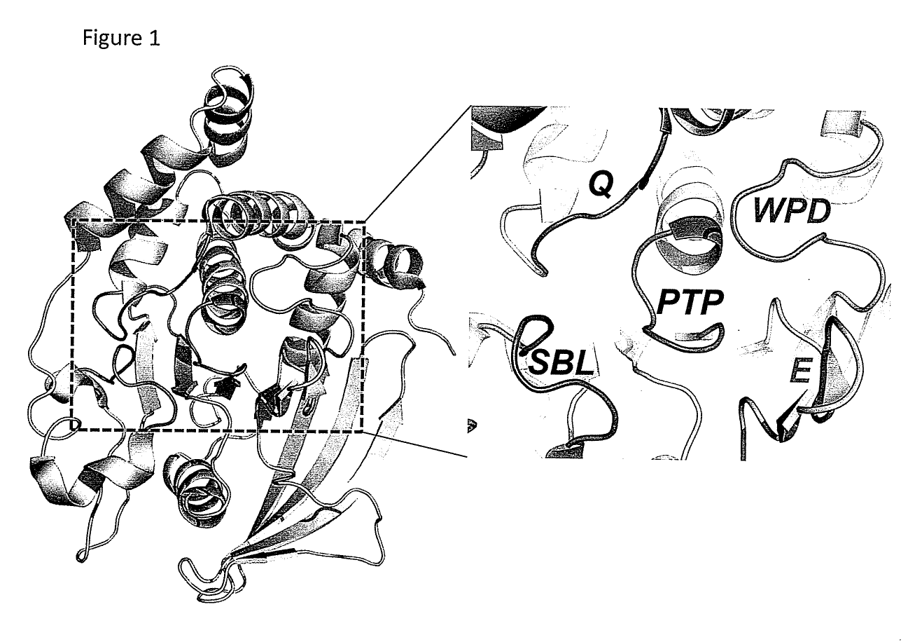

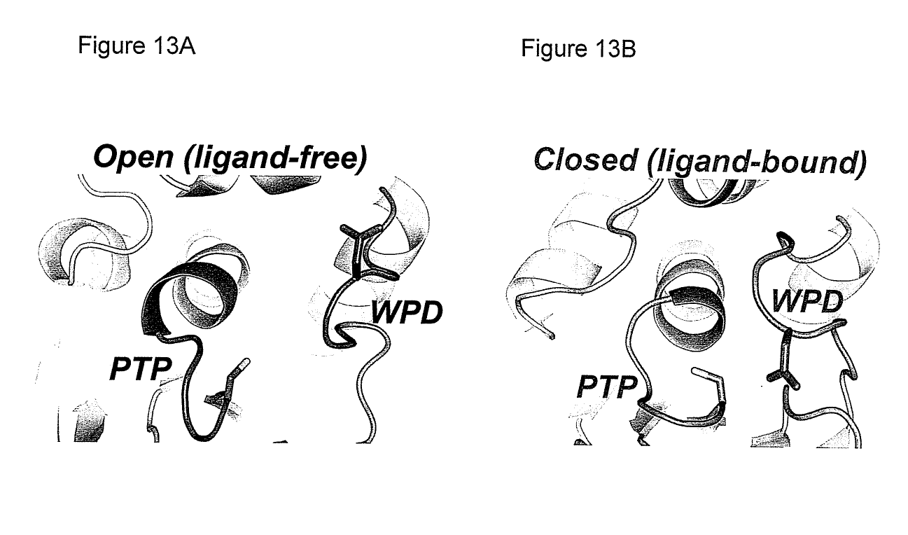

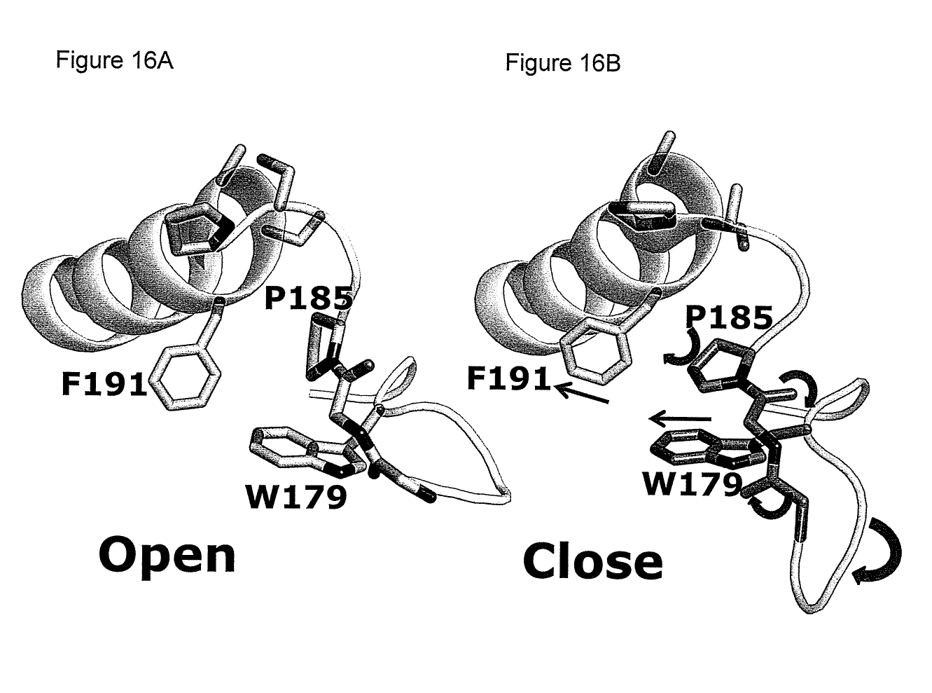

The structure of PTP1B₁₋₃₀₁ was analyzed using x-ray crystallography. See, FIG. 1. The structure was observed to have a size of 1.9 Å. An open/close mechanism regulates k_{cat} because ligand-free PTP1B₁₋₃₀₁ was in an open conformation, and ligand-bound PTP1B₁₋₃₀₁ was in a closed conformation. See, FIG. 13A and FIG. 13B. W179, P185, and F191 were observed to change conformation upon binding of the enzyme with a ligand. See, FIG. 16A and FIG. 16B.

NMR data were collected on Bruker AvanceII 500 and AvanceIIIHD 850 MHz spectrometers equipped with TCI HCN Z-gradient cryoprobes at 298 K. NMR measurements of PTP1B were recorded using either ²H, ¹⁵N- or ²H, ¹⁵N, ¹³C-labeled protein at a final concentration of 0.2-0.5 mM in 50 mM HEPES pH 6.8/7.5, 150 mM NaCl, 0.5 mM TCEP and 90% H2O/10% D₂O. The sequence-specific backbone assignment of PTP1B_{cat} bound by an inhibitor, PTP1B_{Δ7} (e.g. SEQ ID NO: 3) and of PTP1B mutants (N193A, Y152A/Y153A, L192A, T178A) was achieved using the following experiments at 850 MHz ¹H Larmor frequency: 2D [¹H,¹⁵N] TROSY, 3D TROSY-HNCA, 3D TROSY-HN(CO)CA, 3D TROSY-HNCACB and 3D TROSY-HN(CO)CACB. Assignment and titration spectra were processed with Topspin 3.1 (Bruker, Billerica, Mass.) and data were evaluated using SPARKY (http://www.cgl.ucsf.edu/home/sparky/). Relaxation spectra were processed and evaluated using NMRPIPE (http://spin.niddk.nih.gov/NMRPipe/).

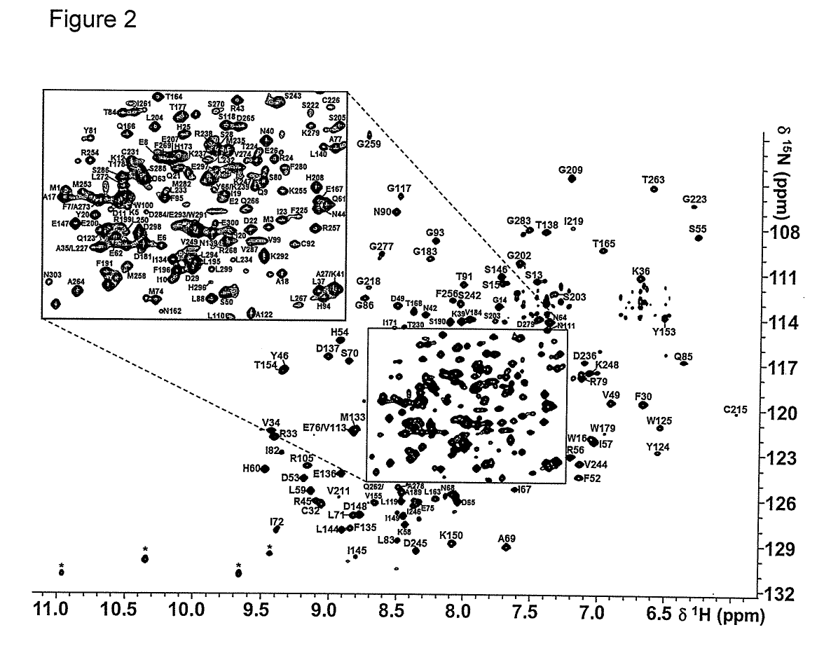

The amino acid sequence of ligand-free PTP1B₁₋₃₀₁ backbone was analyzed by 2D [¹H, ¹⁵N] TROSY NMR spectrometry. See. FIG. 2. TROSY versions of T₁ and T₂ experiments were acquired with a recycle delay of 3 seconds between experiments, and the following relaxation delays for T1: 100, 600, 1000, 1600, 1800, 2000, 2400, and 3000 ms; and T₂: 3.6, 7.2, 14.4, 21.6, 25.2, 28.8, 32.4, 36.0, 61.2, and 72.0 ms. Error shown for both T₁ and T₂ experiments was determined by error in relaxation rate curve fitting.

Seventy-eight percent of the amino acid sequence of the backbone was assigned by ¹⁵NH, and 86% was assigned by ¹³Cα/¹³Cβ. The inset represents 7 out of 8 amino acid residues assigned in the WPD loop, 8 out of 13 amino acid residues assigned in the helix α3, 17 out of 19 amino acid residues assigned in the helix α6, and 7 out of 10 amino acid residues assigned in the helix α7.

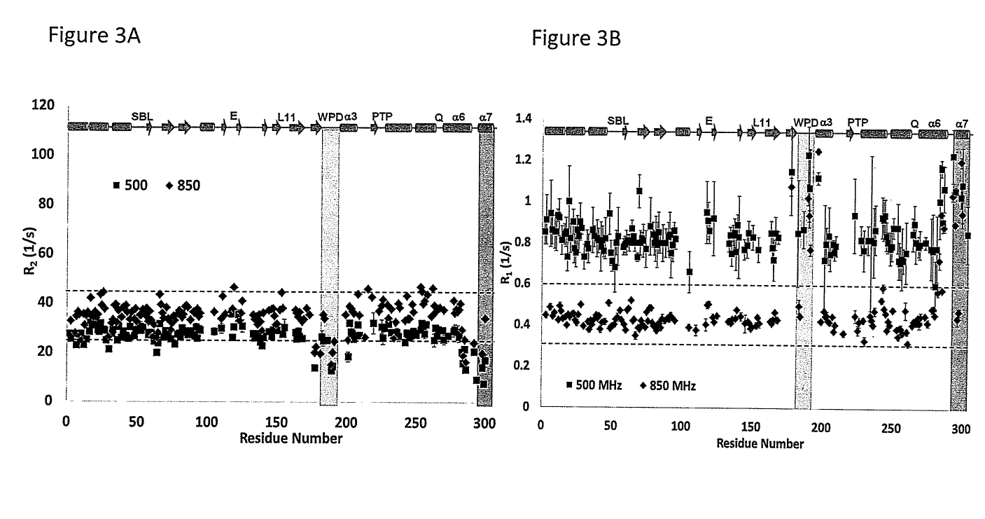

The backbone dynamics of PTP1B₁₋₃₀₁ were analyzed using ¹⁵N NMR to measure nuclear relaxation time. See, FIG. 3A and FIG. 3B. PTP1B₁₋₃₀₁ in apo form had an average T₁ of 2.34 seconds and an average T₂ of 27.75 milliseconds. T₂ and R₁ both show increased ps-ns motions in the WPD loop, helices α3, and helix α7 in the apo state. PTP1B₁₋₃₀₁ at a concentration of 0.2 mM was combined with 50 mM HEPES, 150 mM NaCl, and 0.5 mM of reducing agent TCEP adjusted to pH 6.8. See, FIG. 3A and FIG. 3B. The NMR spectra were recorded at 500 MHz and at 850 MHz with TCI HCN z-gradient cryoprobe at 298 K. Ibid. R₁ calculated at 850 MHz assigned 163 of 226 peaks including 4 out of 8 amino acid residues in the WPD loop, 6 out of 13 in helix α3, 12 out of 19 in helix α6, and 4 out of 10 in helix α7. See, FIG. 3B. R₂ calculated at 850 MHz assigned 170 out of 226 peaks: 5 out of 8 amino acid residues in the WPD loop, 5 out of 13 amino acid residues in the helix α3; 10 out of 19 amino acid residues in the helix α6, and 3 out of 10 amino acid residues in the α7. Ibid. FIG. 3A.

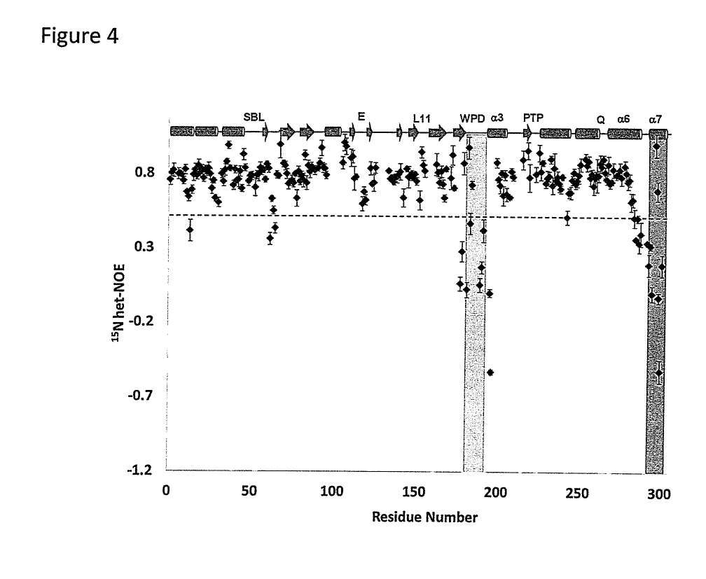

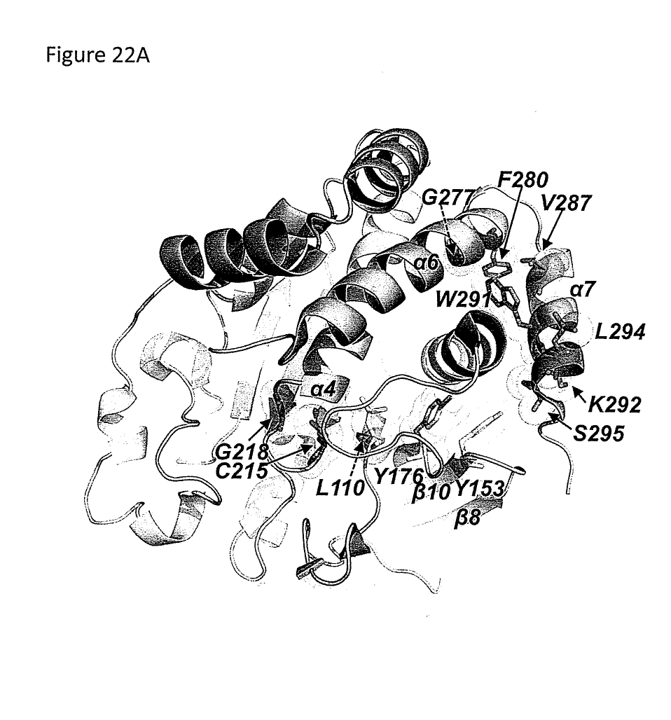

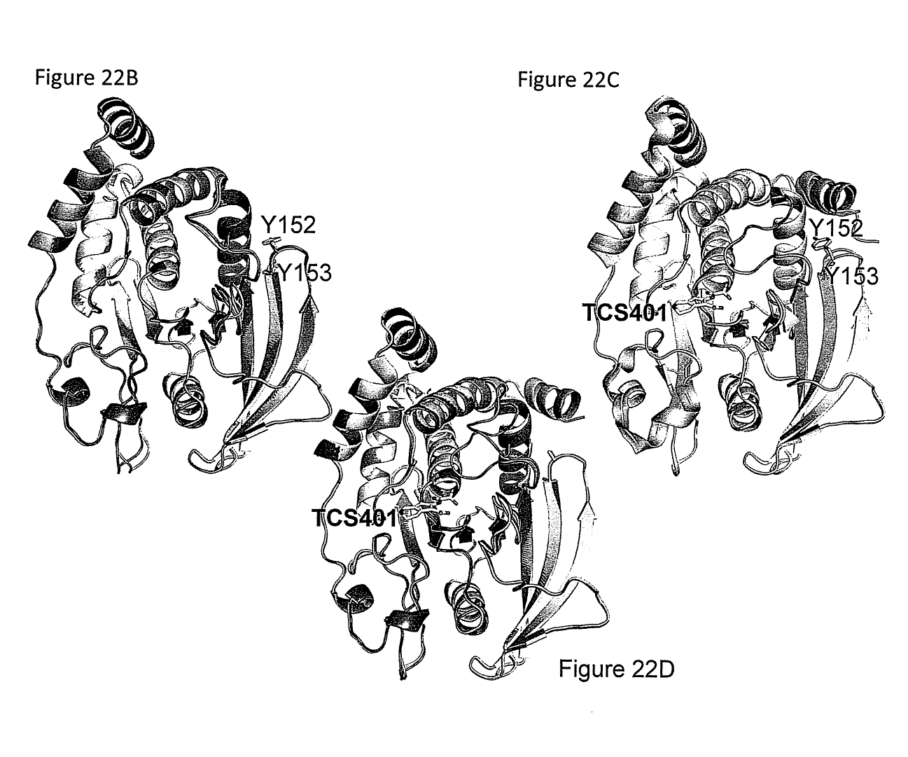

Motions of defined domains of PTP1B₁₋₃₀₁ were analyzed by ¹⁵N [¹H] het-NOE NMR spectroscopy. See, FIG. 4. PTP1B₁₋₃₀₁ at a concentration of 0.2 mM was combined with 50 mM HEPES, 150 mM NaCl, and 0.5 mM of reducing agent TCEP adjusted to pH 6.8. The NMR spectrum was recorded at 500 MHz with TCI HCN z-gradient cryoprobe at 298 K and using 5 second saturation. Ibid. Leu110, Tyr153, Tyr176, Cys215, Gly218, Phe280, Gly277, Val287, Lys292, Leu294, Ser295 were hypothesized to be a functional dynamic spine of PTP1B. See, FIG. 22A.

Higher ps-ns timescale motions were observed in the WPD loop and helix α7 of PTP1B₁₋₃₀₁ compared to the motions measured in other domains of the enzyme. Given that the WPD loop was observed to be responsible for k_{cat}, the effects of an active site or allosteric site inhibitor on PTP1B_{cat} reveal distal effects, particularly on α7 helix. The WPD loop was observed to be forced into a single state, closed with the active site inhibitor and open with the allosteric site inhibitor. Additionally, mutations made in the WPD loop and in regions regulating WPD loop exchange have been observed to affect k_{cat}. The absence of a change in k_{cat} would mean that particular mutation has no noticeable effect on the WPD loop.

### Example 5

### Analysis of Active Site Inhibitor Binding to PTP1B₁₋₃₀₁

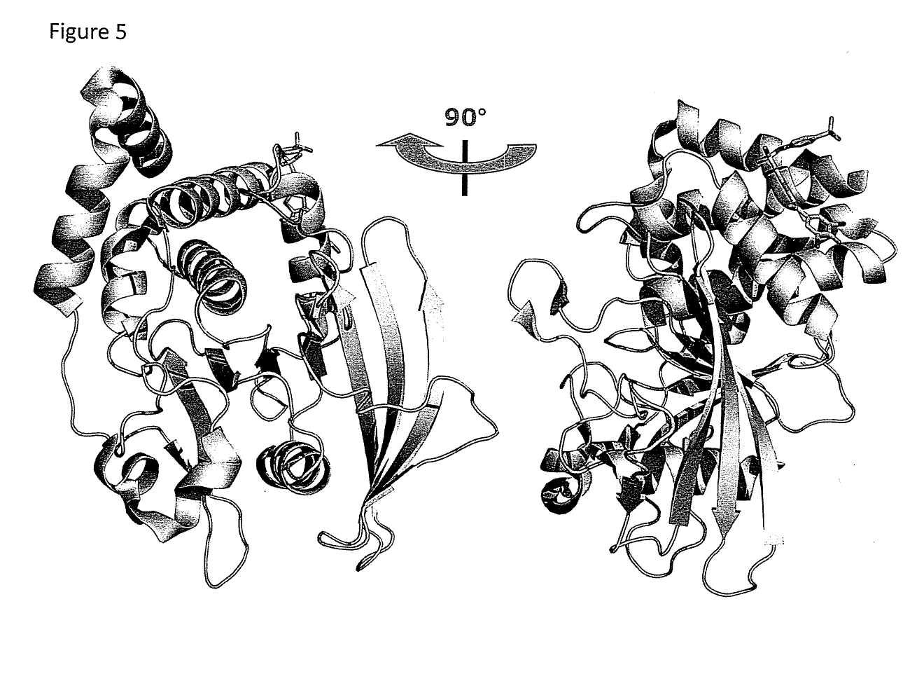

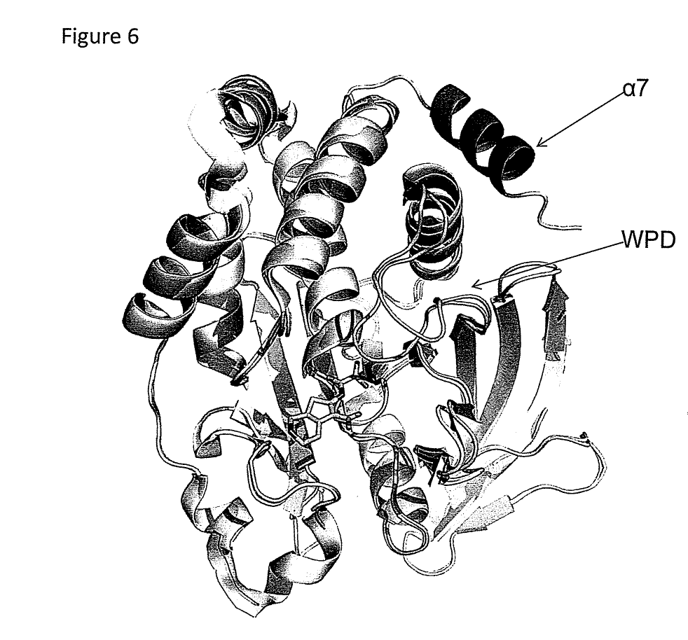

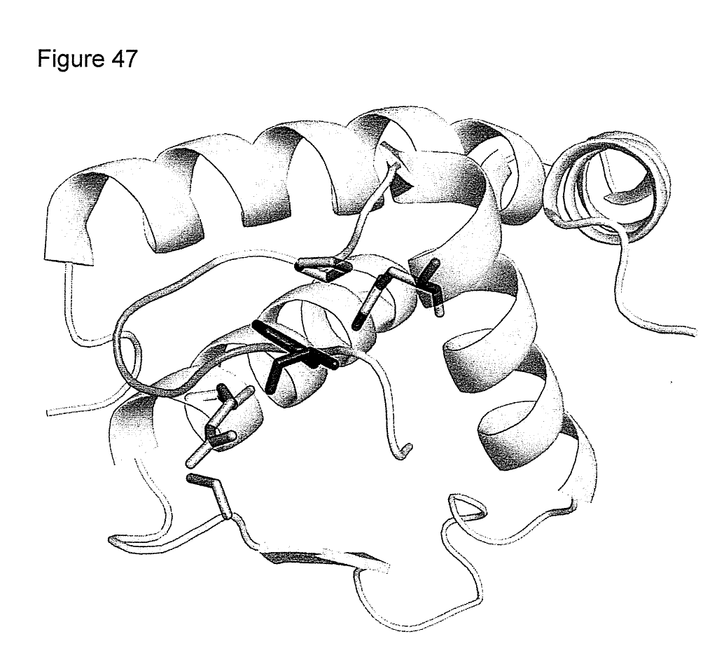

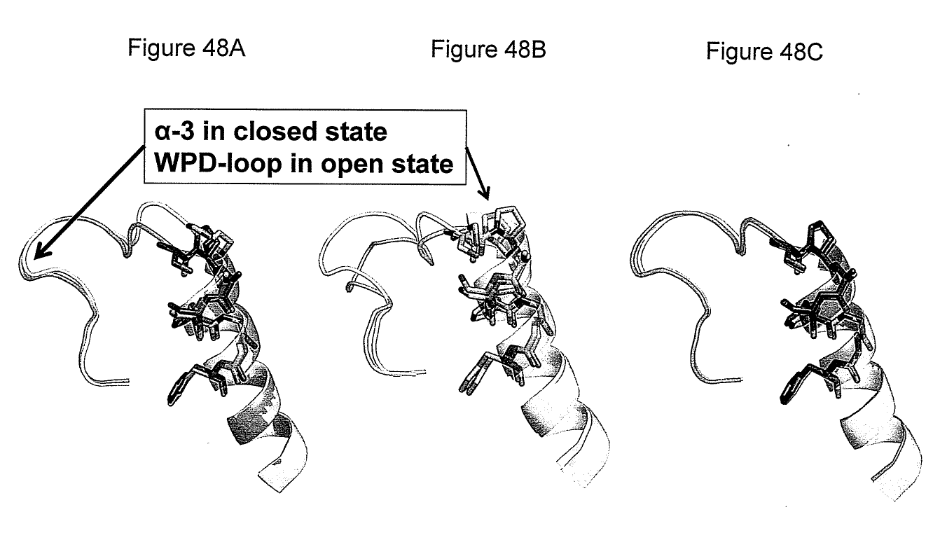

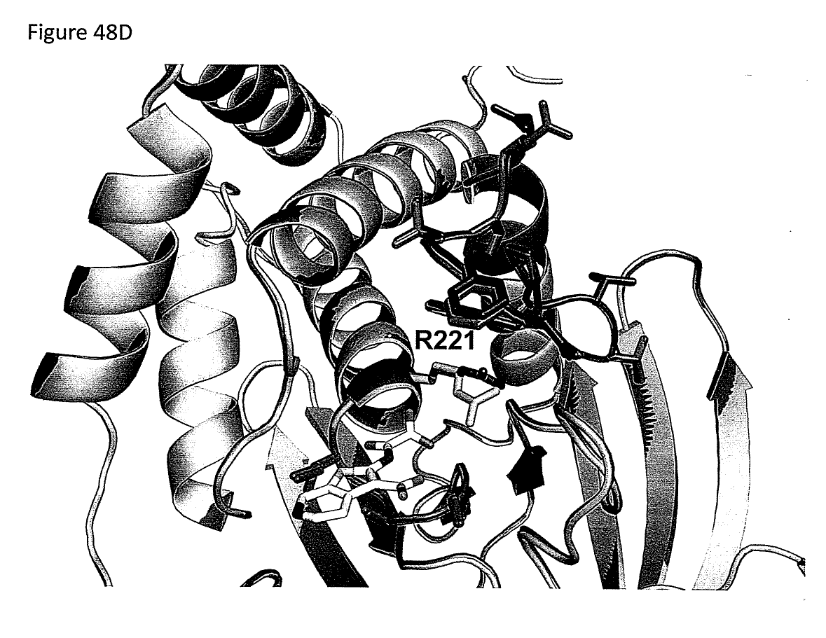

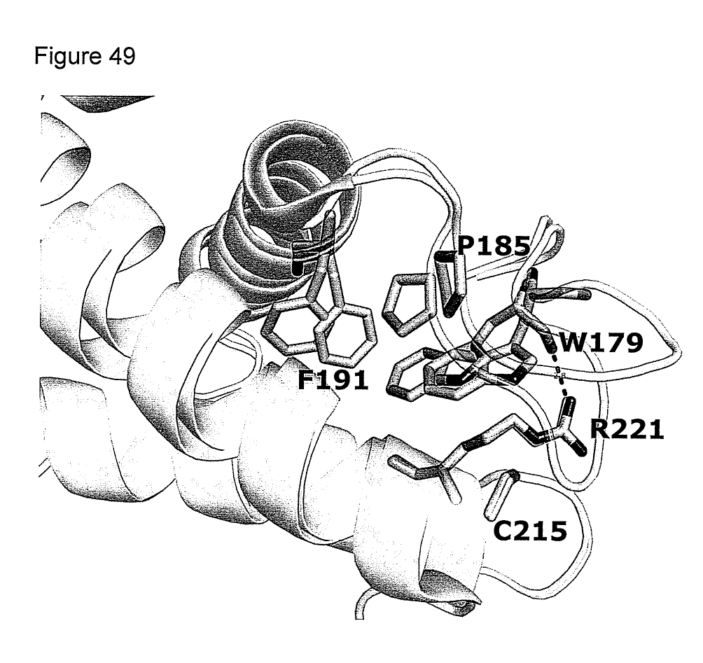

The structure of PTP1B₁₋₃₀₁(PDB: 2HNP) bound to active site inhibitor PDB: 1C88 was analyzed using x-ray crystallography. See, FIG. 6. The structure of amino acid residues 5-282 underwent a structural rearrangement upon binding by PDB: 1C88, and was observed to have a root mean square deviation (RMSD) of 0.41 Å. See, FIG. 6. The structure is similar to that of apo PTP1B₁₋₃₀₁, differing at the WPD loop, which is open in apo state and closed in bound state, and at helix α7. See, FIG. 1. Helix α7 was observed to have no density in apo PTP1B₁₋₃₀₁ which is a common feature of x-ray structures of PTP1B with open WPD loops. WPD loop contains two loops shown in FIG. 47 that have been observed to be active in conformation changes of PTP1B.

Inhibitors were titrated into 200 μM ¹⁵N-PTP1B at molar ratios of 1:1; 1:2, 1:3, 1:5 and 2D [¹H, ¹⁵N] TROSY spectra at 850 MHz were recorded for each titration point. Inhibitors were solubilized in D₆-DMSO at 25 mM. CS for PTP1B in the 2D [¹H, ¹⁵N] TROSY spectrum caused by the addition of D₆-DMSO were negligible. Chemical shift differences (Δδ) between PTP1B and inhibitor-bound PTP1B (3:1 molar ratio) spectra were calculated using:

$$Δ δ ( ppm ) = \sqrt{{( Δ {δ}_{H} )}^{2} + {( \frac{Δ {δ}_{N}}{10} )}^{2}}$$

Cα, Cβ, N and H^{N} chemical shifts (CS) of PTP1B amino acid residues 282-393 (including helix α7) were analyzed using ASTEROIDS ensemble selections, to determine site-specific conformational sampling. Next, a pool of 10,000 statistical coil conformers was generated using the program Flexible-Meccano side chains were added using SCCOMP47 and CS were calculated for each conformer using SPARTA. See, Bernado et al., Proceedings of the National Academy of Sciences of the United States of America 102, 17002-17007 (2005); Ozenne et al., Bioinformatics 28, 1463-1470 (2012); Eyal et al., J Comput Chem 25, 712-724 (2004); Shen et al., Journal of biomolecular NMR 38, 289-302 (2007). ASTEROIDS was used to select five sub-ensembles, each containing 200 conformers on the basis of the experimental CS following an iterative procedure as previously described. See, Nodet et al., Journal of the American Chemical Society 131, 17908-17918 (2009); Jensen et al., Journal of the American Chemical Society 132, 1270-1272 (2010); Ozenne et al., Journal of the American Chemical Society 134, 15138-15148 (2012). The errors used for the CS in the ASTEROIDS calculations were as follows: Cα and Cβ (0.1 ppm), N (0.2 ppm) and HN (0.04 ppm).

Dihedral angles from the selected structures were extracted for each amino acid and used to build representative conformers of PTP1B₁₋₃₉₃. PTP1B amino acid residues 300-393 were connected to the catalytic domain PTP1B crystal structure (PDBid: 1 SUG) by Flexible-Meccano using the amino acid specific conformational sampling derived by ASTEROIDS, creating a pool of 15000 conformers. This pool contained 3 populations, which differ in the behavior of helix α7. In 5000 conformers, α7 was maintained as identified in 1SUG and PTP1B amino acid residues 300-393 started C-terminal of α7. In 7000 conformers, amino acid residues 285-296 were assumed to be disordered. In 3000 conformers, helix α7 was intact but no longer interacted with the surface of PTP1B. The rational for the selection from these different pools was 1) the initial NMR CS analysis suggested only about 40% helical behavior of α7 and 2) α7 has no corresponding electron density in numerous PTP1B crystal structures.

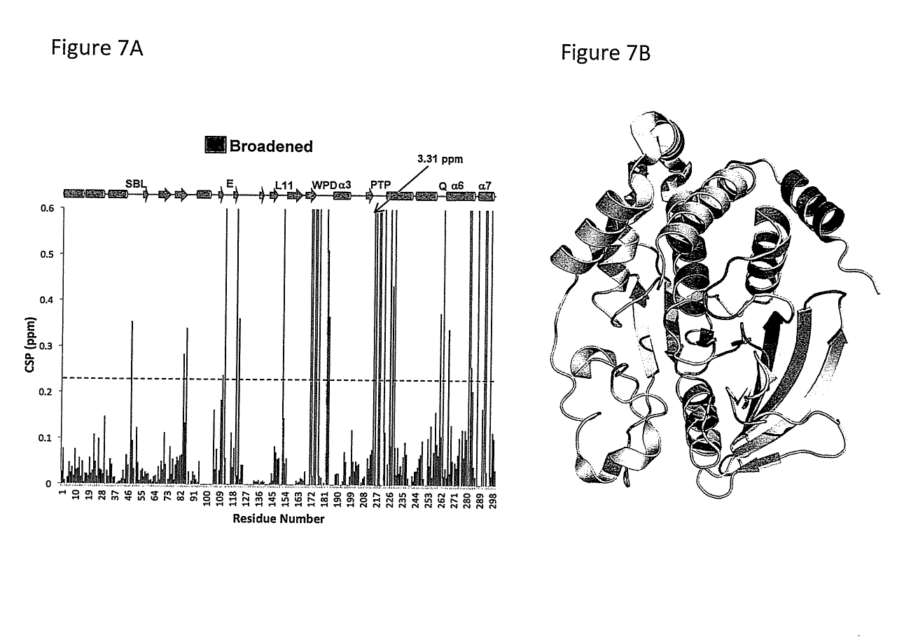

PTP1B₁₋₃₀₁ bound to TCS-401 had CS outside of the active site as indicated by peaks corresponding to amino acid residues in the E loop, L11 loop, and WPD loop. See, FIG. 7A. Analysis of the CS is based on assignment of 74% of the PTP1B backbone by ¹⁵NH, and assignment of 5 out of 8 amino acid residues in the WPD loop, 8 out of 13 in helix α3, 16 out of 19 in helix α6, and 5 out of 10 in helix α7. A ribbon model of PTP1B₁₋₃₀₁ bound to TCS-401 highlighting in red the amino acid residues with CS greater than the mean plus 2σ is shown in FIG. 7B.

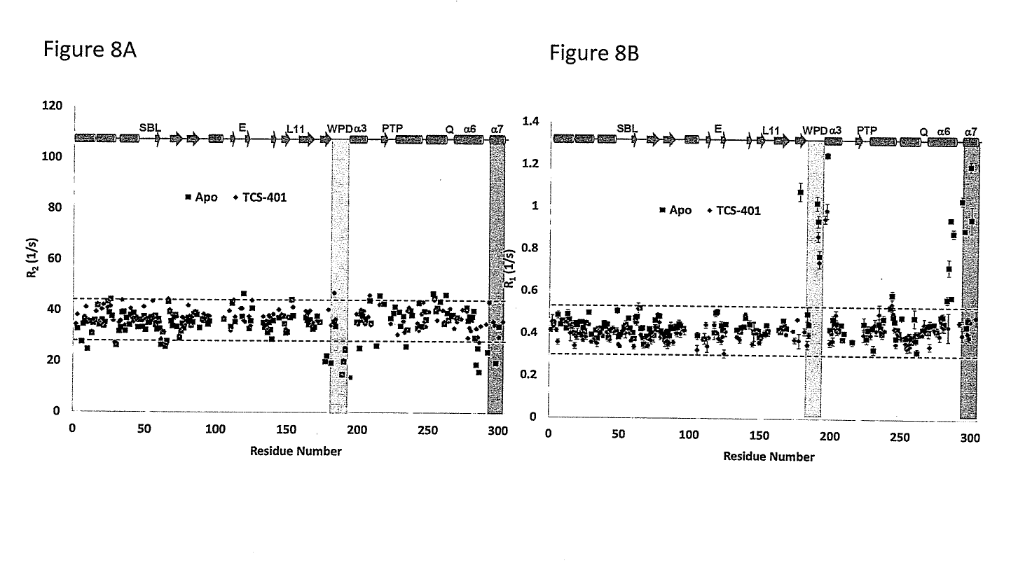

Changes in motions of the domains of PTP1B₁₋₃₀₁ bound to TCS-401 were measured by detecting nuclear relaxation time data at 500 MHz. See, FIG. 8A and FIG. 8B. Fast-timescale backbone dynamics were measured for ligand-free and inhibitor-bound (at 3:1 molar ratios) PTP1B constructs at either 850 or 500 MHz 1H Larmor frequency. ¹⁵N longitudinal (R₁) and transverse (R₂) relaxation rates and ¹⁵N [¹H]-NOE (hetNOE) measurements were acquired. The hetNOE measurements were determined from a pair of interleaved spectra acquired with or without pre-saturation, and a recycle delay of 5 seconds (only at 500 MHz ¹H Larmor frequency). Error was determined using signal to noise ratio for a given peak.

In FIG. 8A, 163 of 214 peaks were assigned amino acid residues. Four of 8 amino acid residues in the WPD loop, 7 out of 13 in the α3 helix, 11 out of 19 in the α3 helix, and 5 out of 10 in the α7 helix were assigned. See, FIG. 8A. Active site inhibitor binding was observed to lead to a loss of motions in the α7 helix and part of the WPD loop as compared to apo PTP1B₁₋₃₀₁ in a 1:3 ratio of PTP1B₁₋₃₀₁ to TCS-401, according to R₂ values. See, FIG. 8A.

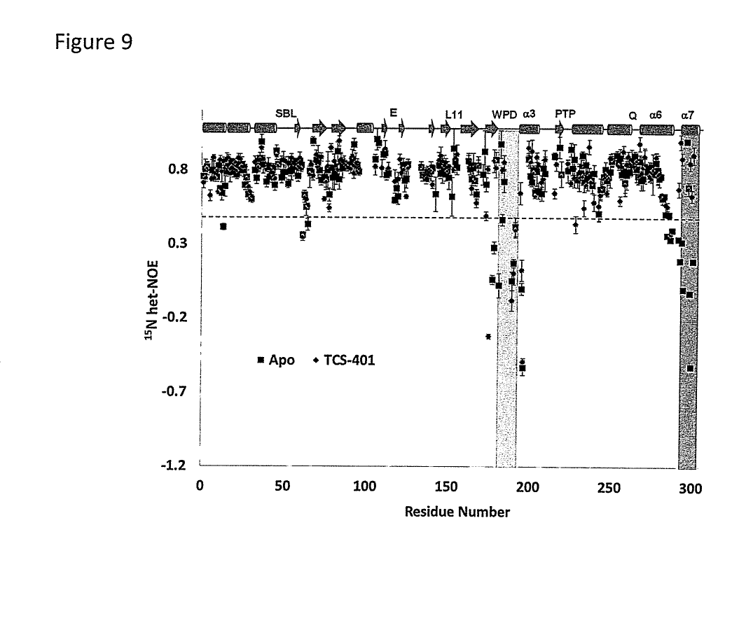

In FIG. 8B, 154 of 214 peaks were assigned amino acid residues. Four of 8 amino acid residues in the WPD loop, 8 out of 13 in the α3 helix, 12 out of 19 in the α3 helix, and 2 out of 10 in the α7 helix. See, FIG. 8B. R₁ values also indicate that active site inhibitor binding leads to a loss of motions in the α7 helix and part of the WPD loop as compared to apo PTP1B₁₋₃₀₁ in a 1:3 ratio of PTP1B₁₋₃₀₁ to TCS-401. See, FIG. 8B. ¹⁵N het-NOE results at 500 MHz confirm the above conclusion. See, FIG. 9.

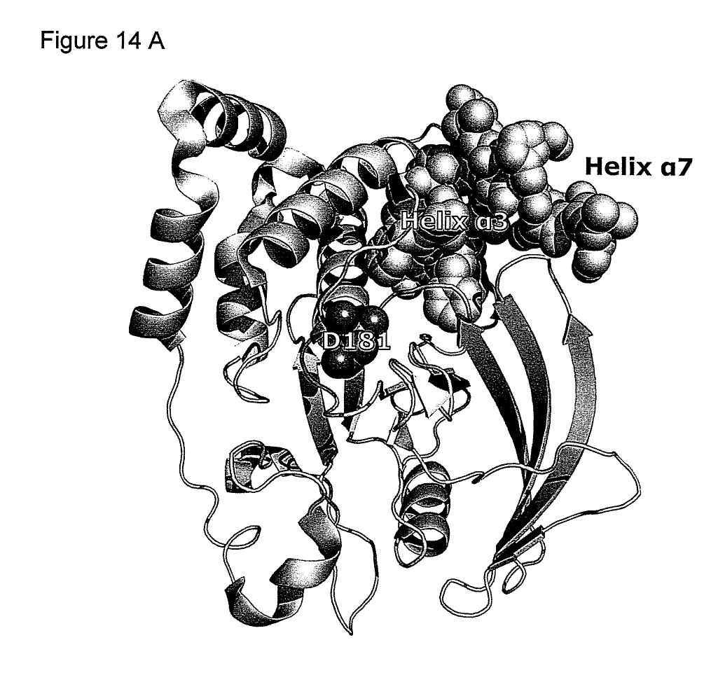

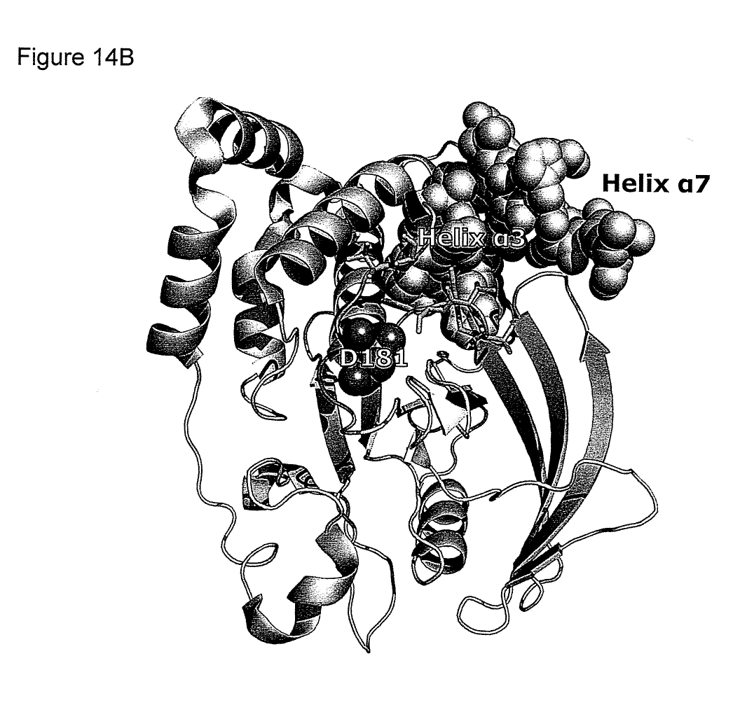

X-ray crystallography was used to analyze changes in structure between PTP1B₁₋₃₀₁ bound to active inhibitor TCS-401 and apo PTP1B₁₋₃₀₁. Ribbon models of PTP1B₁₋₃₀₁ with the α3 helix, the α7 helix, and D181 labeled illustrate changes in conformation resulting from binding with active site inhibitor TCS-401. See, FIG. 14A and FIG. 14B.

In contrast to both the apo and allosteric bound states, ps-ns motions in the n-terminal portion of the WPD loop and helix α7 (distal from the active site) were observed to be lost upon binding of the TCS-401 active site inhibitor. Therefore, the catalytic domain at α7 helix was truncated to determine effects of α7 helix on the ps-ms motions of PTP1B.

### Example 6

### Analysis of Allosteric Inhibition of PTP1B₁₋₃₀₁

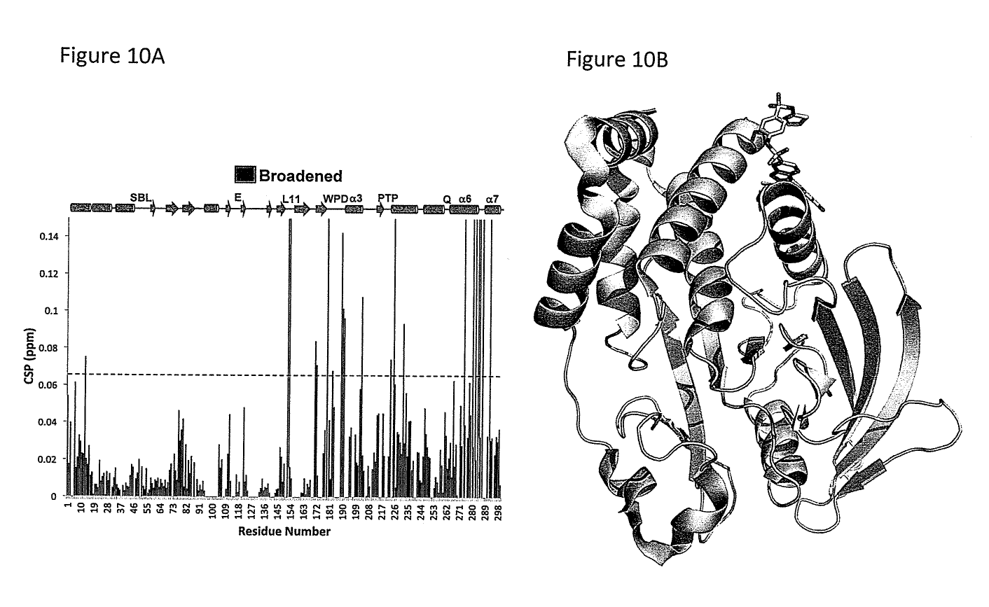

The structure of PTP1B₁₋₃₀₁ bound to allosteric inhibitors PDB: 1T49 and PDB: 1T4J were analyzed using x-ray crystallography. See, FIG. 5 and FIG. 10B.

CS analysis at 850 MHz resulted in assignment of 74% of the PTP1B backbone by ¹⁵NH, and assignment of 6 out of 8 amino acid residues in the WPD loop, 8 out of 13 in helix α3, 16 out of 19 in helix α6, and 4 out of 11 in helix α7. See, FIG. 10A. The binding caused CS in PTP1B₁₋₃₀₁ bound to PDB: 1T4J at the L11 and the WPD loop. The complete sequence specific backbone assignment was confirmed by complete sequence specific backbone assignment in the presence of a 3 molar surplus of inhibitor

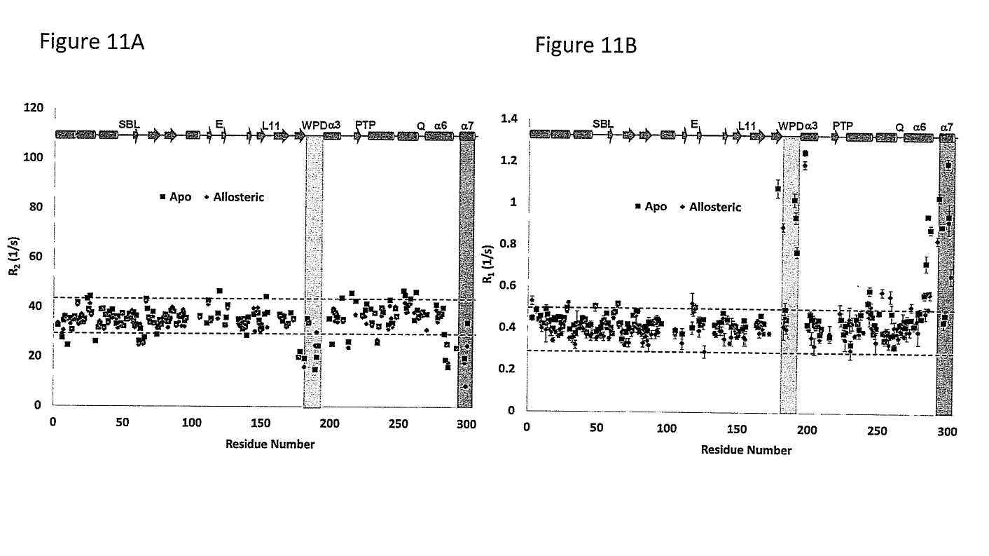

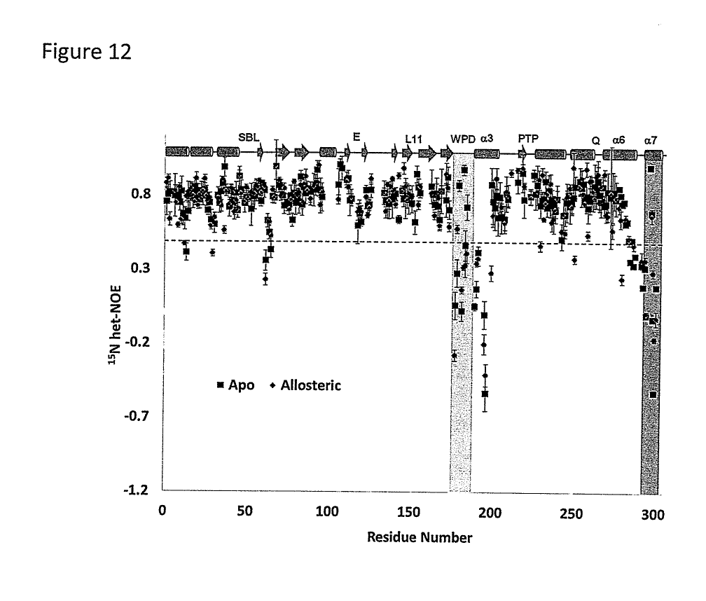

Changes in motions of the domains of PTP1B₁₋₃₀₁ bound to TCS-401 were measured by detecting nuclear relaxation time data at 850 MHz. See, FIG. 11A and FIG. 11B. In FIG. 11A, 149 of 219 peaks were assigned, as well as 4 out of 8 amino acid residues in the WPD loop, 2 out of 13 amino acid residues in the α3 helix, 11 out of 19 amino acid residues in the α6 helix, and 3 out of 10 in the α7 helix were assigned. In FIG. 11B, 146 of 219 peaks were assigned amino acids, as well as 3 out of 8 amino acid residues in WPD loop, 3 out of 13 amino acid residues in the α3 helix, 9 out of 19 amino acid residues in the α6 helix, and 3 out of 10 amino acid residues in the α7 helix. In the allosteric inhibitor bound state, ps-ns backbone dynamics were observed to be similar to those of the apo state. See, FIG. 11A and FIG. 11B. ¹⁵N-hetNOE titration of amino acid residues 5-282 at 500 MHz confirmed this conclusion. See, FIG. 12.

Therefore, allosteric site inhibitor binding was observed to have minimal effect on PTP1B₁₋₃₀₁ dynamics.

### Example 7

### Analysis of the Role of Helix α7 in PTP1B

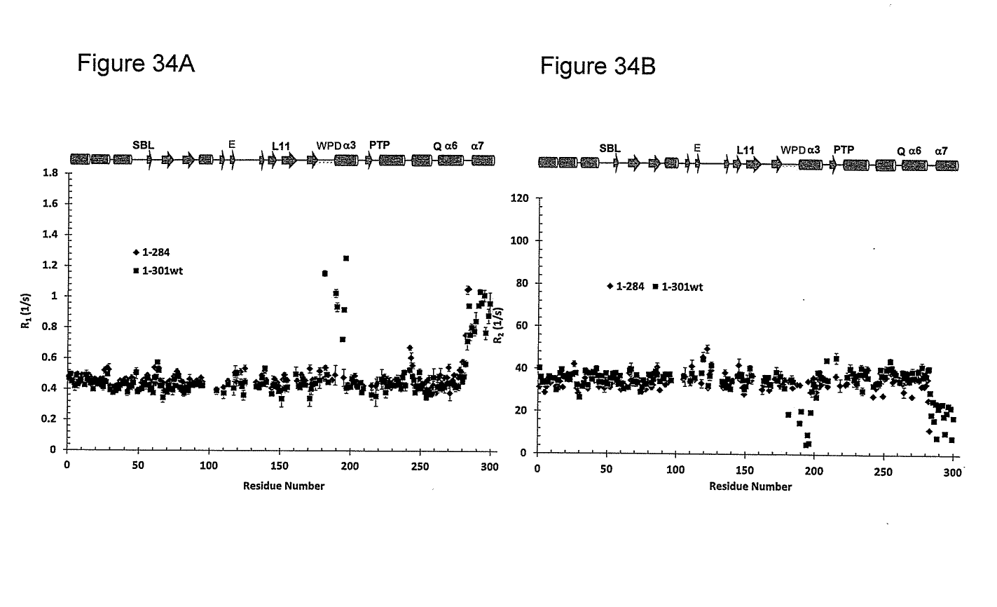

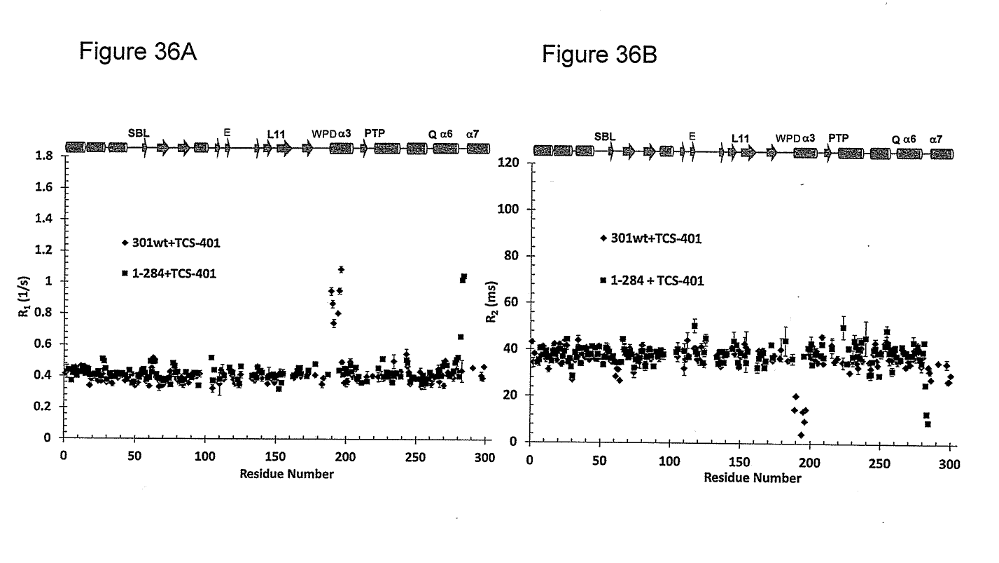

The differences in dynamics for each domain of wild-type PTP1B₁₋₃₀₁ (gray) to PTP1B₁₋₂₈₄ (black) were compared at 850 MHz using overlays of NMR nuclear relaxation time data. See, FIG. 34A and FIG. 34B. FIG. 34A is an overlay of R₁ (1/s) values for wild-type PTP1B₁₋₃₀₁ and PTP1B₁₋₂₈₄. FIG. 34B is an overlay of R₂ (1/s) values for wild-type PTP1B₁₋₃₀₁ and PTP1B₁₋₂₈₄. Further differences were observed by comparing NMR nuclear relaxation time data of wild-type PTP1B₁₋₃₀₁ bound to TCS-401 (gray) to PTP1B_{Δ7} bound to TCS-401 (black). See, FIG. 36A and FIG. 36B.

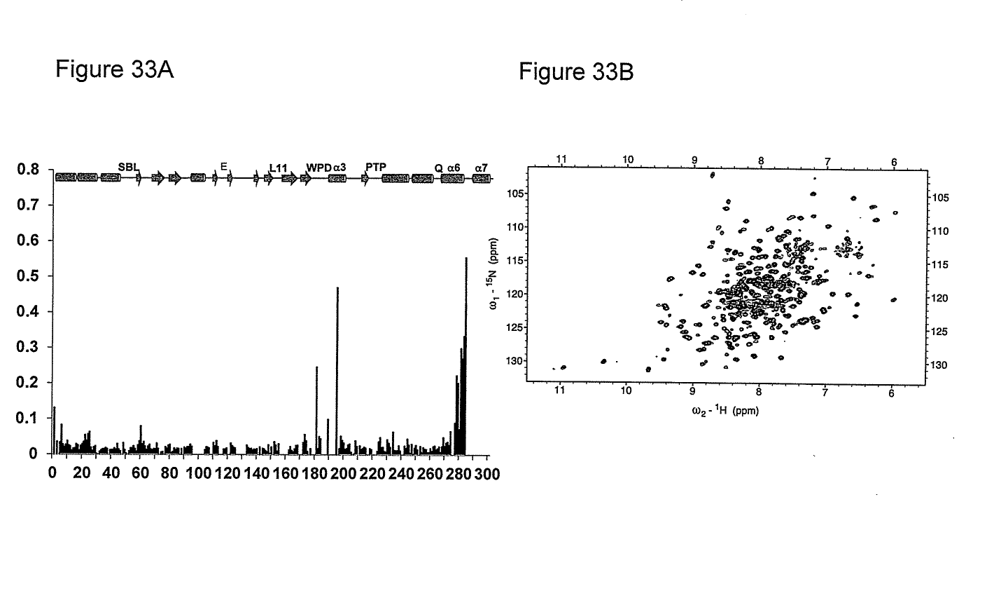

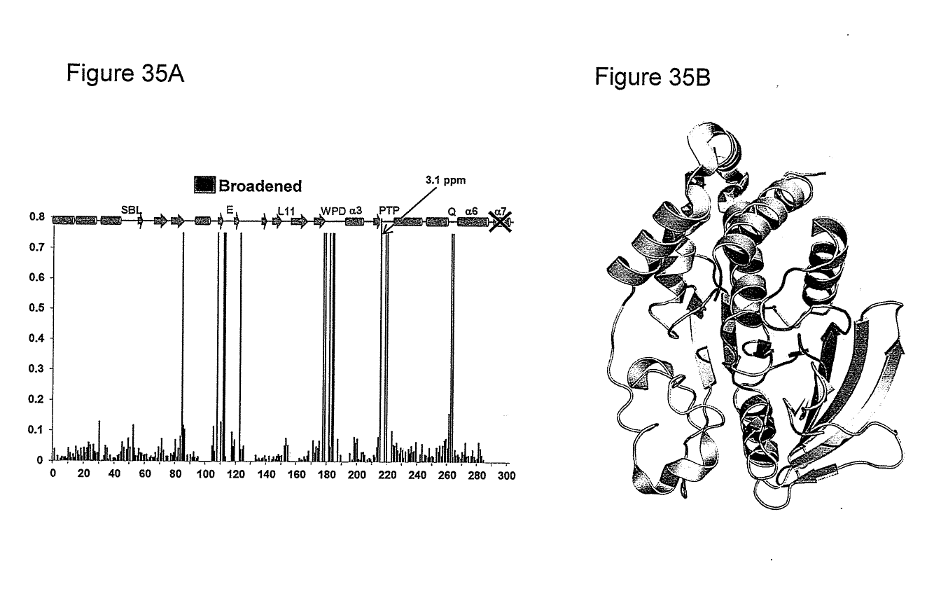

PTP1B₁₋₂₈₄ bound to TCS-401 was observed to have greater CS than PTP1B₁₋₂₈₄ alone. See, FIG. 33A and FIG. 35A. FIG. 35B is a ribbon model of PTP1B_{Δ7} bound to TCS-401 highlighting in red the amino acid residues with CS greater than the mean plus 2σ. 2D [¹H, ¹⁵N] TROSY NMR spectra of wild-type PTP1B₁₋₃₀₁ and PTP1B₁₋₂₈₄ were observed to vary. See, FIG. 33B.

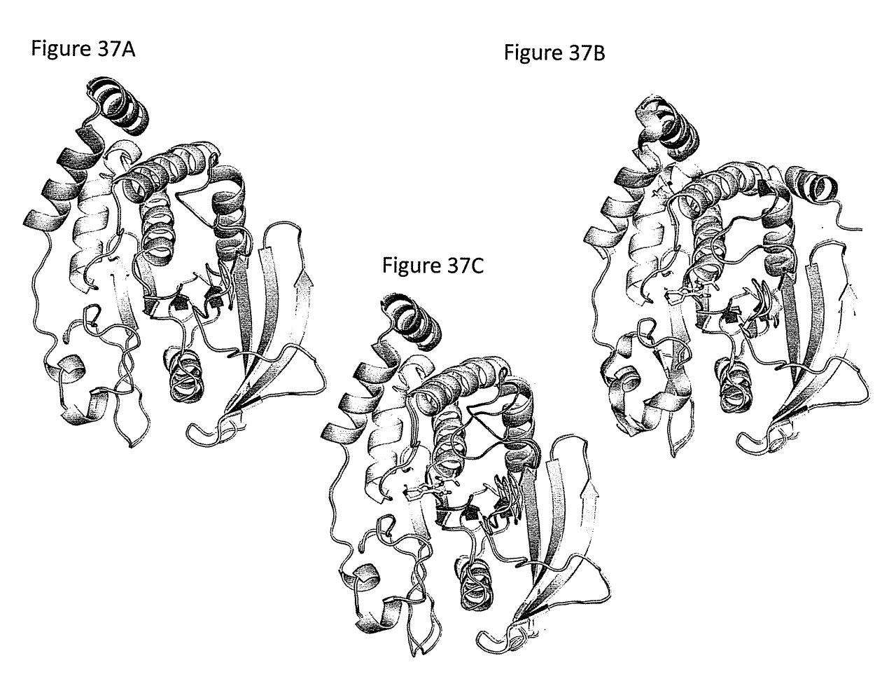

Structural variations were observed among wild-type PTP1B, wild-type PTP1B bound to TCS-401, PTP1B₁₋₂₈₄ bound to TCS-401, and PTP1B₁₋₂₈₄ based on x-ray crystallography data. See, FIG. 37A, FIG. 37B, and FIG. 37C.

Deletions at amino acid residues that were not hypothesized herein to be involved in the interaction of α7 helix with the catalytic domain were observed to have no effect on k_{cat}. The WPD loop became more ridged on the ps-ns timescale in PTP1B₁₋₂₈₄. Despite the changes in the ps-ns motions, the truncation of the α7 helix was not observed to affect the fold of the catalytic domain, and the WPD loop was observed to remain in the open conformation in the absence of the α7 helix.

### Example 8

### Analysis of the Role of the L11 Loop in PTP1B Activity Regulation Using Mutant PTP1B_{1-301 YAYA}

PTP1B_{YAYA} is a mutant of PTP1B₁₋₃₀₁ having point mutations at Y152 and Y153 within the L11 loop. The structure of PTP1B_{YAYA} was analyzed using x-ray crystallography. See, FIG. 22B. In the ribbon model, the active site/PTP loop is cyan, and the WPD is red X-crystallography, and the location of the mutations, Y152 and Y153, is orange. PTP1B_{YAYA} was observed to have a size of 2.1 Å according to x-ray crystallography.

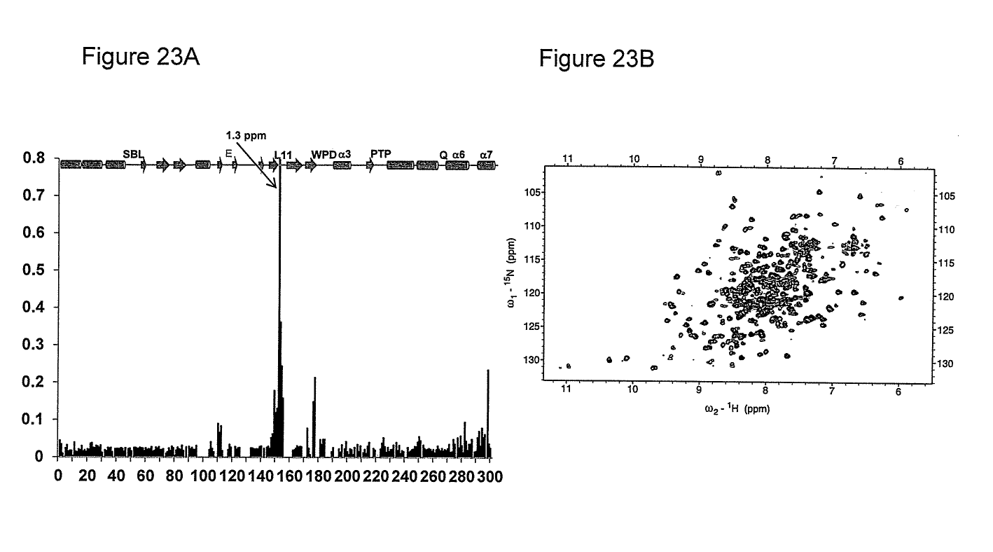

CS analysis of PTP1B₁₋₃₀₁ and PTP1B_{YAYA} resulted in the highest peak at 1.3 ppm located within the L11 loop. See, FIG. 23A. 2D [¹H, ¹⁵N] TROSY NMR spectrometry was performed at 850 MHz to compare the backbones of ligand-free PTP1B₁₋₃₀₁ (red) and PTP1B_{YAYA} (blue). See, FIG. 23B.

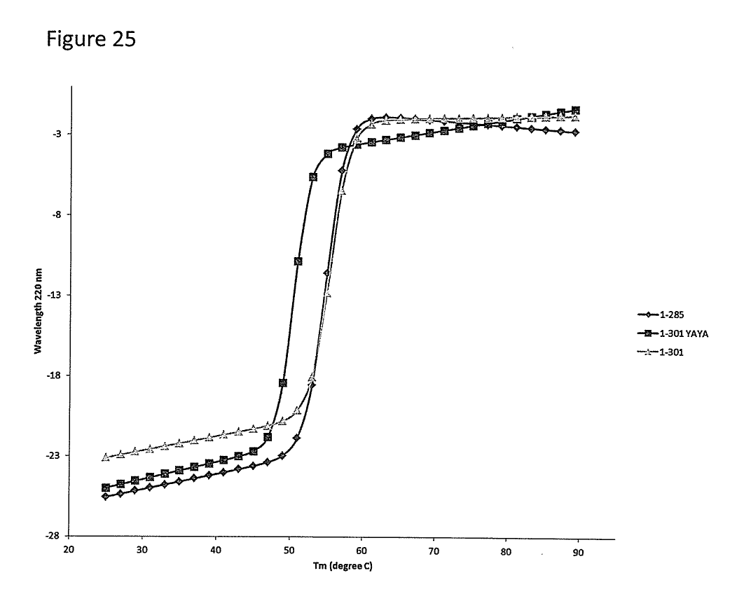

Phosphatase activity of PTP1B against the general PTP/DUSP substrate, p-nitrophenyl phosphate (pNPP), was tested in 50 mM HEPES pH 7.5, 150 mM NaCl, 0.5 mM TCEP. 40 μl of enzyme at a 1.5 μM concentration was added to 60 μl of pNPP substrate resuspended in the reaction buffer. See, FIG. 25. The final concentrations of pNPP used in this experiment were 0, 0.5, 1, 2, 4, and 8 μM. The enzyme was incubated with the substrate at 30° C. for 30 min, after which 100 μl of 1 M NaOH was added to a final concentration of 0.5 M NaOH to quench the reaction. Absorbance was measured at 405 nm and the final concentration of p-nitrophenol (pNP) was calculated using the molar extinction coefficient of 18000 M-1 cm-1. A control reaction in which substrate was incubated in the absence of enzyme was subtracted from all other experimental values. All experiments were performed in triplicate.

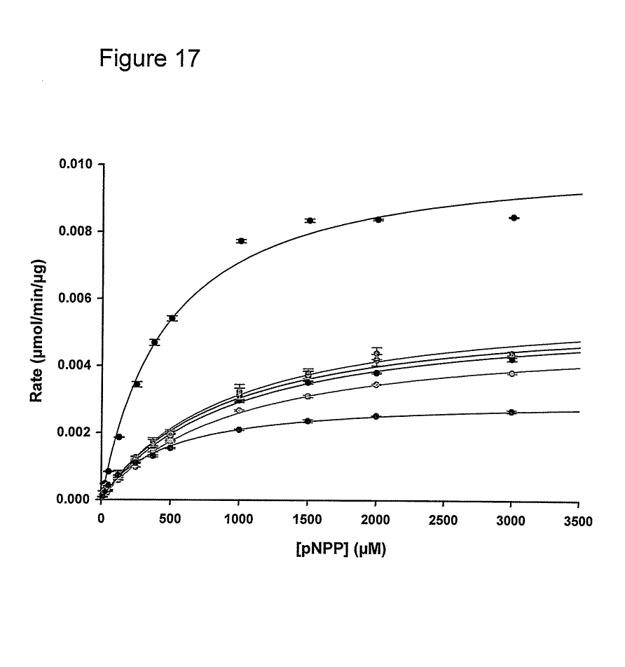

Enzyme assays were performed at pH 7.5 with 0.15 μM PTP1B to compare the activities of wild-type PTP1B₁₋₃₀₁ to mutants with mutations in the L11 loop: PTP1B_{Y152A/F}, PTP1B_{Y153A/F}, and PTP1B_{YAYA}. See, FIG. 17. Wild-type PTP1B₁₋₃₀₁ (blue) was observed to have a K, of 0.46±0.06 mM, k_{cat} of 6.2±0.3 s⁻¹, and a melting point (Tₘ) of 55.46° C. Ibid. PTP1B_{Y153A} (teal) was observed to have a Kₘ of 0.93±0.18 mM, k_{cat} of 3.6±0.29 s⁻¹, and a Tₘ of 53.75° C. Ibid. PTP1B_{Y152A} (orange) was observed to have a Kₘ of 0.96±0.04 mM, k_{cat} of 3.0±0.06 s⁻¹, and a Tₘ of 53.89° C. Ibid. PTP1B_{Y153F} (purple) was observed to have a Kₘ of 0.90±0.08 mM and a k_{cat} of 3.4±0.3 s⁻¹. Ibid. PTP1B_{Y152F} (green) was observed to have a Kₘ of 0.95±0.04 mM and a k_{cat} of 3.4±0.1 s⁻¹. Ibid. PTP1B_{YAYA} (red) was observed to have a Kₘ of 0.45±0.04 mM, k_{cat} of 1.8±0.05 s⁻¹, and a Tₘ of 50.41° C. Ibid.

PTP_{YAYA} exhibited similar activity to PTP1B₁₋₂₈₄, indicating L11 links helix α7 to the active site. See, FIG. 25. Additional mutants (Y176, T177, T178) based on the CS data herein were used to verify the link.

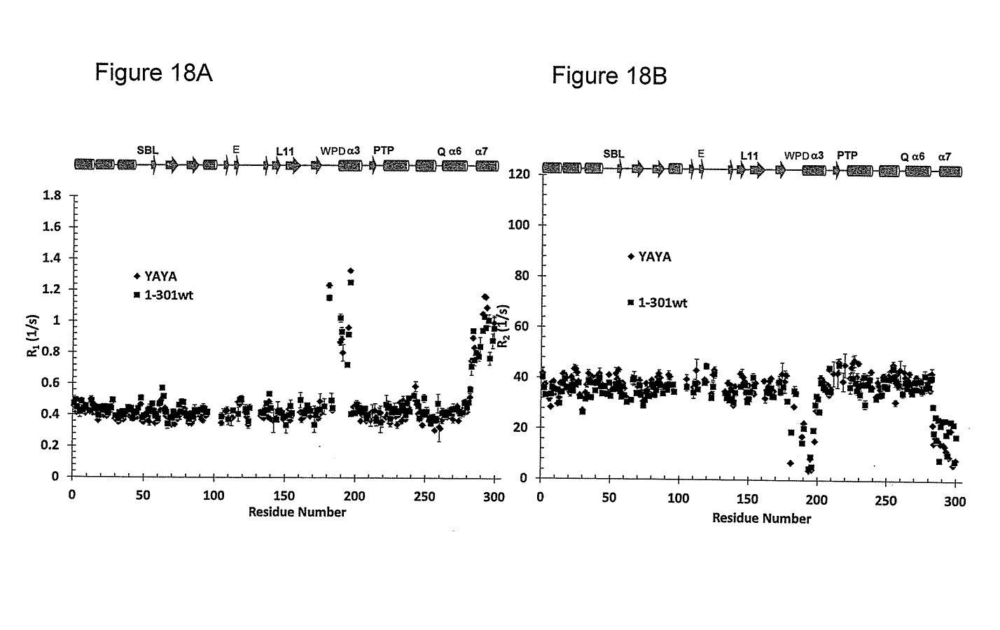

Motions of the domains of PTP1B₁₋₃₀₁ (black) and PTP1B_{YAYA} (gray) were compared by detecting nuclear relaxation time data at 850 MHz. See, FIG. 18A and FIG. 18B. R₁ (1/s) values are shown in FIG. 18A. R₂ (1/s) are shown in FIG. 18B.

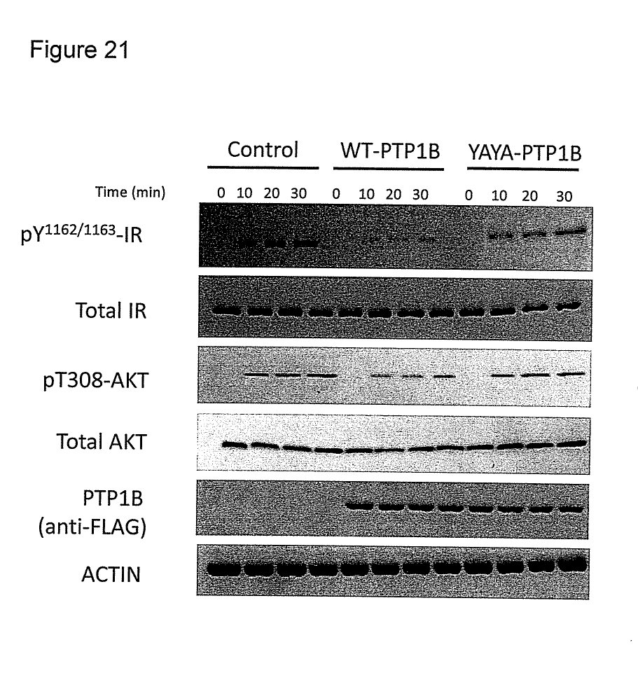

HEK293 cells were transfected with control (vector only), wild-type PTP1B₁₋₃₀₁, or PTP1B_{YAYA} and incubated for 24 hours. Cells were serum starved for 10 hours and stimulated with insulin (25 nM) for 0, 10, 20 and 30 minutes. After stimulation, cells were lysed, and lysates were used to analyze the effect of PTP1B_{YAYA} on insulin signaling. FIG. 21 is a photograph of a gel showing the results.

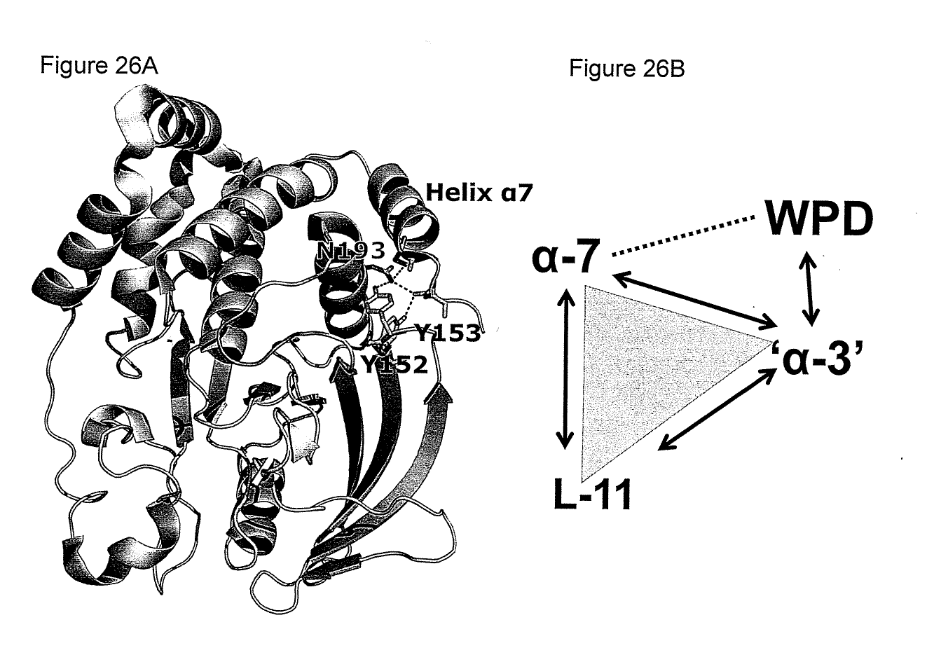

The L-11 loop was observed to directly interact with the α3 helix and the α7 helix, and indirectly interact with the WPD loop through the α3 helix. See, FIG. 26B.

A ribbon model of the dynamic spine of PTP1B based on x-ray crystallography analysis is shown in FIG. 22A. The α7 helix is shown in green, the PTP/active site is shown in pink, and the E loop, the WPD loop, and the L11 loop are shown in yellow. Labeled amino acids F280, V287, W291, L294, K292, G218, Y176, Y153, L110, C215, and 5295 were observed to regulate catalytic activity.

Circular dichroism (CD) spectropolarimetry was used to measure the melting temperatures of PTP1B_{YAYA}, PTP1B₁₋₃₀₁, and PTP1B₁₋₂₈₄. CD measurements were performed on a Jasco J-815 CD spectropolarimeter. PTP1B samples were dialyzed at 4° C. into 10 mM sodium phosphate, pH 6.8, 150 mM NaCl. Samples were then diluted to a protein concentration of 2.5 μM and placed in a 0.2-cm glass cuvette. Wavelength scans were performed in the far-UV region from 260 to 195 nm at 25° C. at a speed of 20 nm/min. Thermal denaturation scans were performed at 208 nm from 25 to 90° C. at a scan speed of 1° C./min. Raw data were processed using the denatured protein analysis routine implemented in the SepctraAnalysis software and converted to mean residue molar ellipticity using the following equation: [θ]=θ/(10×C×1×n), where θ is ellipticity, C is the molar concentration, 1 is the cell path length in centimeters, and n is the number of residues. Plots are the average of three replicate scans.

The melting temperature for PTP1B_{YAYA} (50.41° C.±0.090) was observed to be about 5° C. lower than for PTP1B₁₋₃₀₁ (55.46° C.±0.098) and about 4.5° C. lower than PTP1B₁₋₂₈₄ (54.87° C.±0.094), indicating that decreased activity is due to decreased stability.

### Example 9

### Analysis of PTP1B_{1-301 YAYA} Bound to Active Site Inhibitor TCS-401

PTP1B_{YAYA} bound to TCS-401 was observed to have a size of 2.1 Å according to x-ray crystallography analysis.

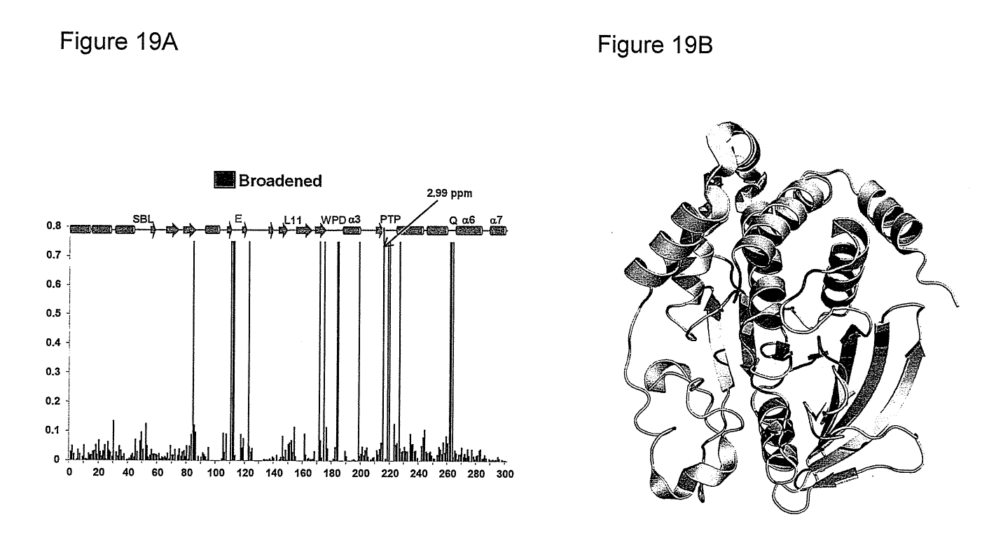

PTP1B_{YAYA} bound to TCS-401 had CS as measured at 850 MHz. See, FIG. 19A. The highest peak (2.99 ppm) was located in the PTP/active site. Ibid. The analysis resulted in assignment of 77% of the backbone using ¹⁵NH, 4 out of 10 amino acid residues in the WPD loop, 8 out of 11 amino acid residues in the α3 helix, and 11 out of 12 amino acid residues in the α7 helix. In a ribbon model based on x-ray crystallography data, the residues with CS are highlighted in red with backbone spheres in the background of the model. See, FIG. 19B. TCS-401 was observed to affect dynamics of the PTP1B backbone.

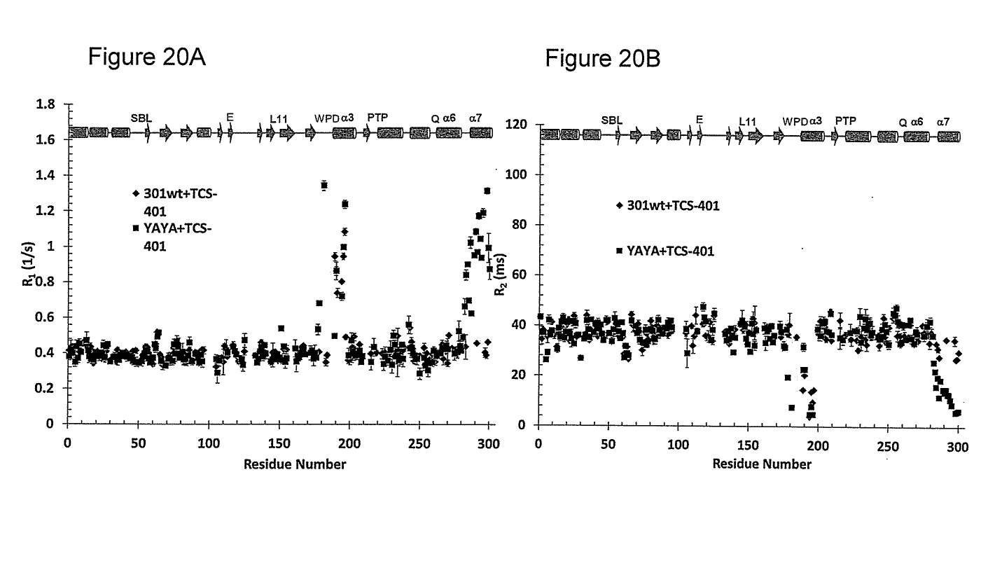

Motions of the domains of PTP1B₁₋₃₀₁ bound to TCS-401 (gray) and PTP1B_{YAYA} bound to TCS-401 (black) were compared by detecting nuclear relaxation time data at 500 MHz for each protein. See, FIG. 20A and FIG. 20B. R₁ (1/s) values are shown in FIG. 20A. R₂ (1/s) are shown in FIG. 20B.

The structures of wild-type PTP1B₁₋₃₀₁ and PTP1B_{YAYA} are observed to be altered by binding with active site inhibitor TCS-401. See, FIG. 22B, FIG. 22C, and FIG. 22D.

### Example 10

### Role of α3 Helix in PTP1B Activity Regulation

X-ray crystallography was used to determine the structure of PTP1B_{N193A}, which has a mutation in the α3 helix. See, FIG. 26A. In one embodiment, the point mutation occurs in an amino acid sequence substantially similar to SEQ ID NO: 2. PTP1B_{N193A} was observed to have a size of 1.6 Å.

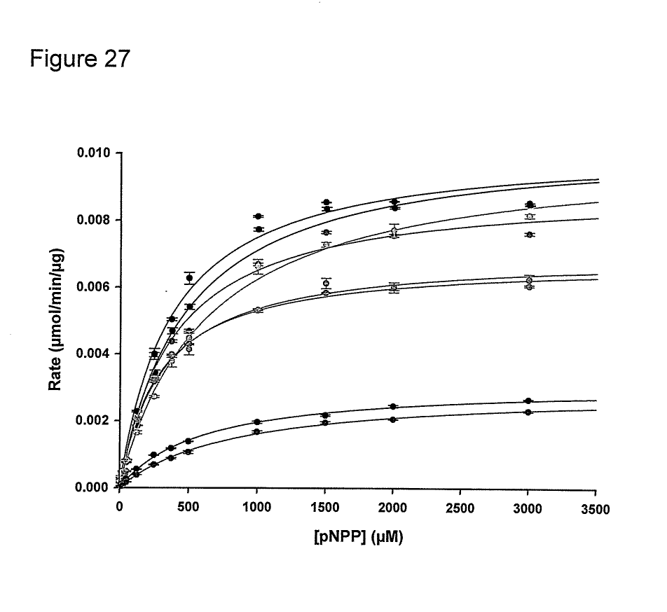

Enzyme assays were performed at pH 7.5 with 0.015 μM PTP1B to compare the activities of wild-type PTP1B₁₋₃₀₁ to mutants with mutations in the α3 helix such as PTP1B_{N193A}. See, FIG. 27. Wild-type PTP1B₁₋₃₀₁ (blue) was observed to have a Kₘ of 0.46±0.06 mM, k_{cat} of 6.2±0.3 s⁻¹, and a melting point (Tₘ) of 55.46° C. Ibid. PTP1B_{S190A} (purple) was observed to have a Kₘ of 0.37±0.05 mM and a k_{cat} of 6.1±0.3 s⁻¹. Ibid. PTP1B_{L192A} (green) was observed to have a Kₘ of 0.61±0.04 mM, k_{cat} of 1.86±0.04 s⁻¹, and a Tₘ of 53.89° C. Ibid. PTP1B_{Y153F} (orange) was observed to have a Kₘ of 0.62±0.07 mM and a k_{cat} of 6.0±0.2 s⁻¹. Ibid. PTP1B_{N193A} (red) was observed to have a Kₘ of 0.83±0.07 mM, a k_{cat} of 1.7±0.06 s⁻¹, and a Tₘ of 49.1° C. Ibid. PTP1B_{F194A} (pink) was observed to have a Kₘ of 0.39±0.05 mM and a k_{cat} of 5.3±0.3 s⁻¹. Ibid. PTP1B_{L195A} (teal) was observed to have a Kₘ of 0.29±0.03 mM and a k_{cat} of 4.1±0.1 s⁻¹. PTP1B_{F196A} (light green) was observed to have a Kₘ of 0.32±0.04 mM and a k_{cat} of 4.2±0.2 s⁻¹.

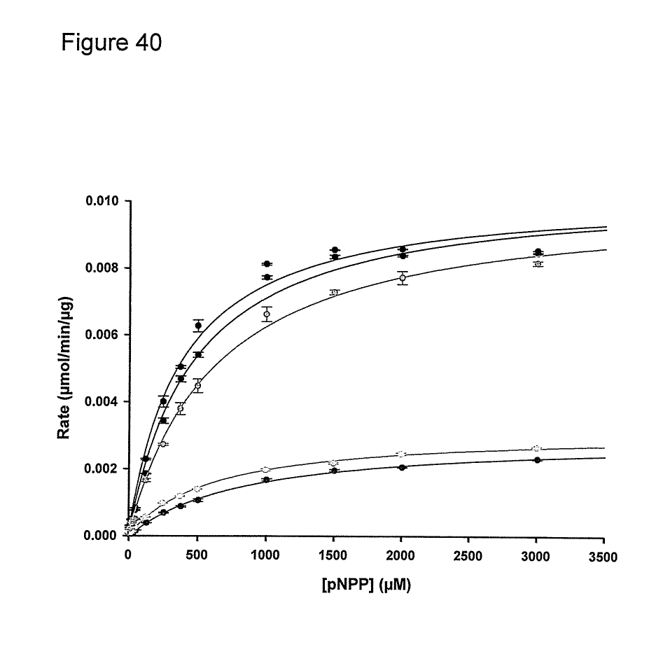

Further, enzyme assays were performed at pH 7.5 at enzyme concentrations of 0.15 for wild-type PTP1B₁₋₃₀₁ and the mutants utilized in FIG. 27. See, FIG. 40. Activity was observed to remain at similar levels at reduced concentrations. See, FIG. 40.

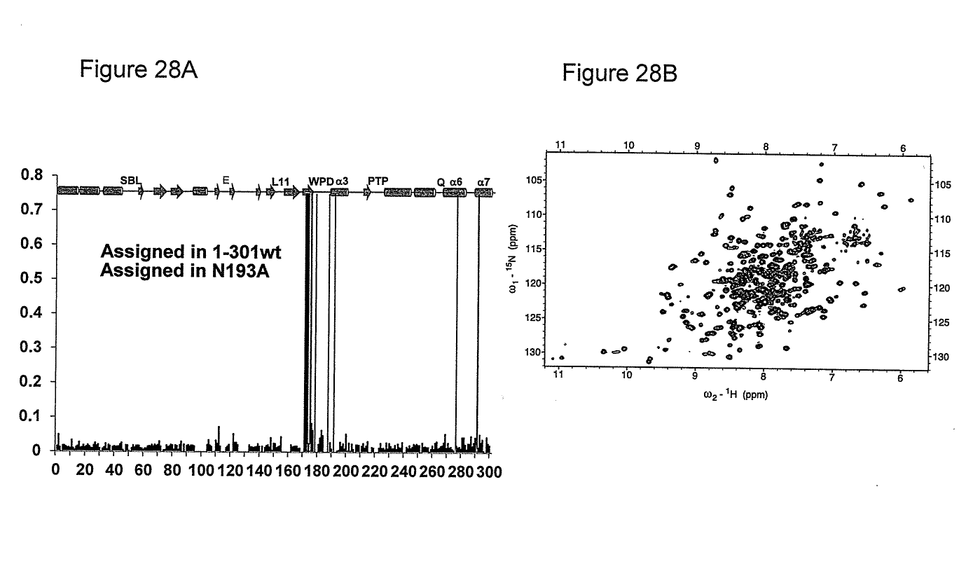

CS analysis at 850 MHz comparing wild-type PTP1B₁₋₃₀₁ (red) and PTP1B_{N193A} (green) indicated CS in the α3 helix and the α6 helix for PTP1B_{N193A}, as compared to the CS for _{wild}-type PTP1B₁₋₃₀₁ which were in the WPD loop and the α7 helix. See, FIG. 28A. FIG. 28B is TROSY spectra used to compare the dynamics of PTP1B₁₋₃₀₁ (red) to PTP1B_{N193A} (blue).

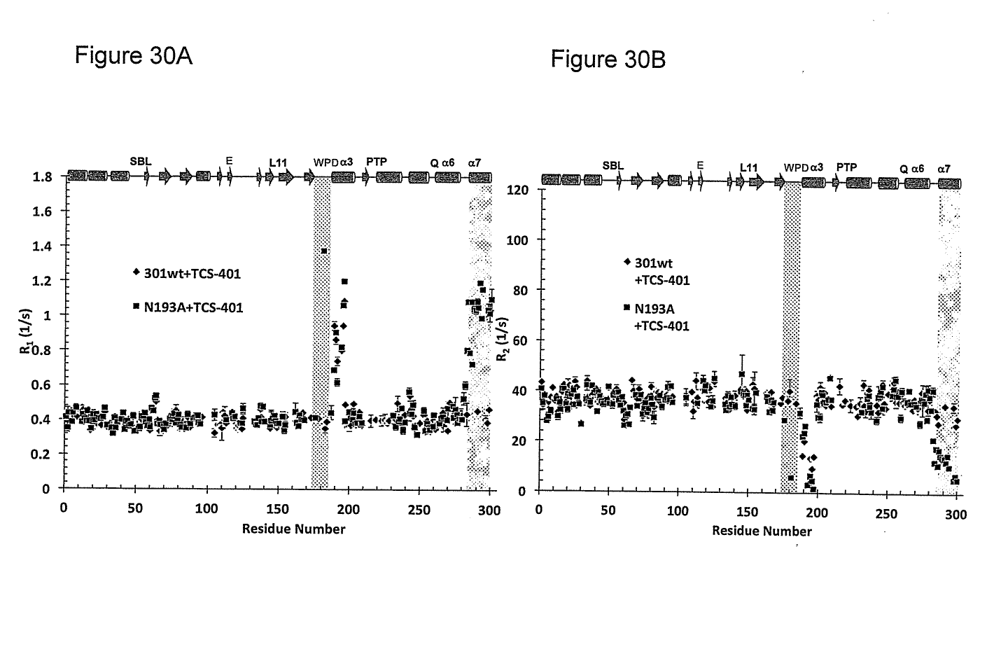

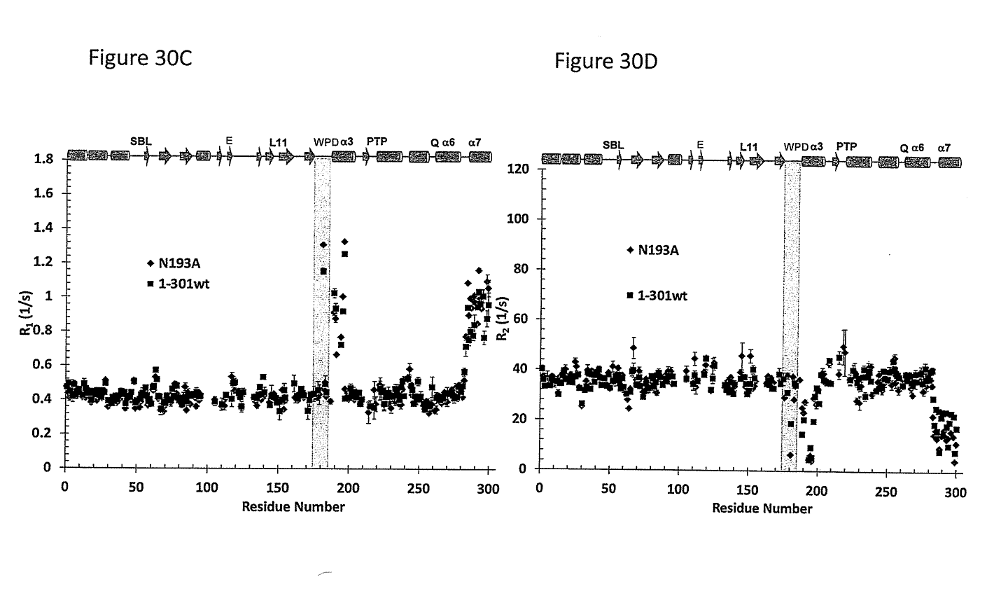

Changes in motions of the domains of wild-type PTP1B₁₋₃₀₁ (black) compared to PTP1B_{N193A} (gray) were observed by nuclear relaxation time data at 850 MHz. See, FIG. 30C and FIG. 30D.

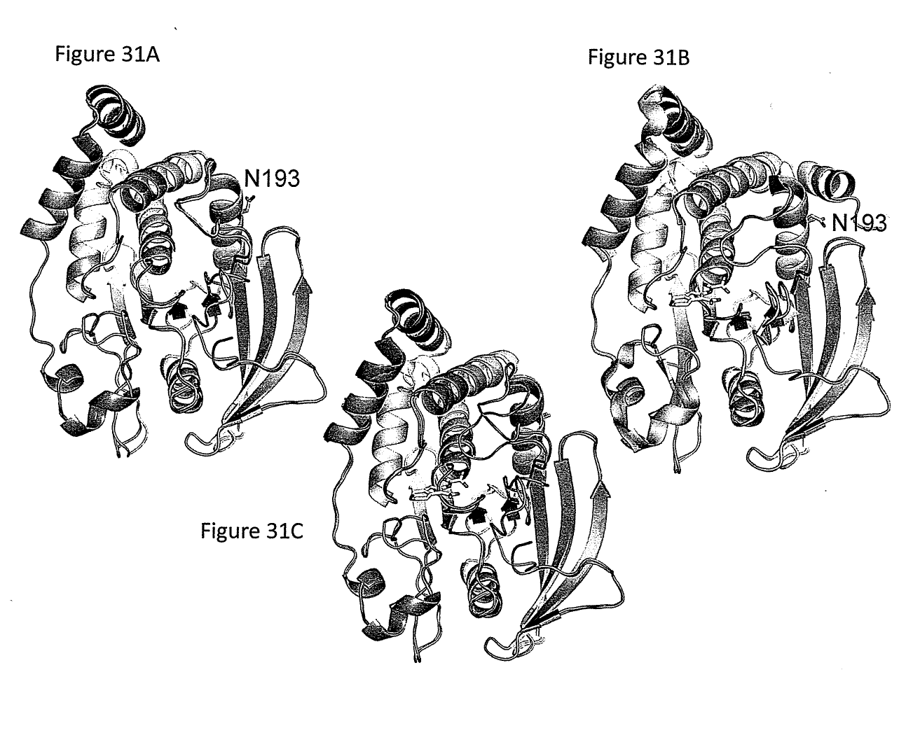

Structural variations were observed by x-ray crystallography among wild-type PTP₁₋₃₀₁, wild-type PTP1B₁₋₃₀₁ bound to TCS-401, PTP1B_{N193A}, and PTP1B_{N193A} bound to TCS-410. See, FIG. 31A, FIG. 31B, and FIG. 31C. PTP_{N1934} bound to TCS-401 was observed to have a size of 1.9 Å based on x-ray crystallography analysis.

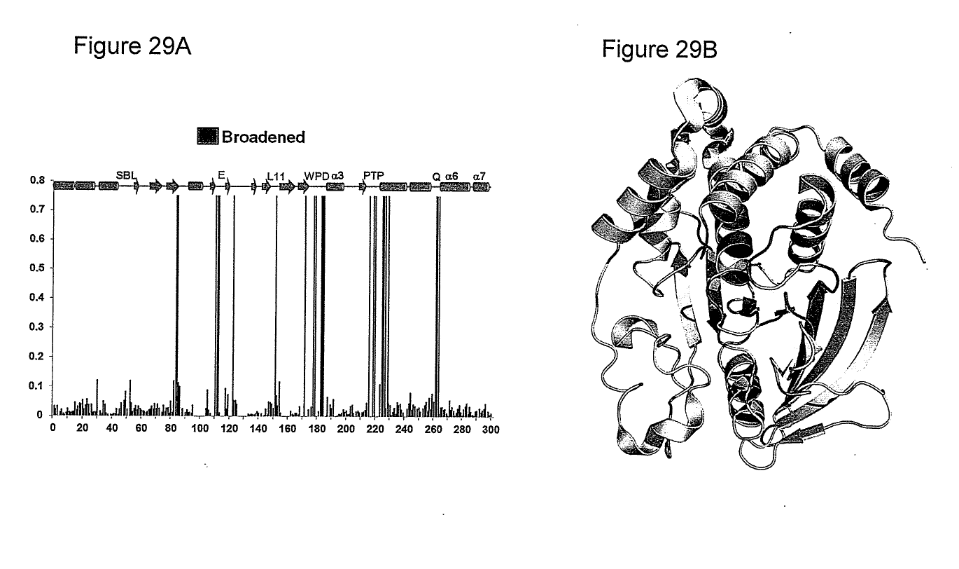

Changes in motions of the domains of wild-type PTP1B₁₋₃₀₁ (black) and were compared to PTP1B_{N193A} (gray) using nuclear relaxation time data gathered at 850 MHz. See, FIG. 30A and FIG. 30B. CS (red) in PTP1B_{N193A} were observed due to TCS-401 binding. See, FIG. 29A. The ribbon model in FIG. 29B illustrates the locations of the amino acid residues with CS greater than the mean plus 2σ.

### Example 11

### Analysis of the Role of α7 Helix in PTP1B Activity Regulation

A truncated protein was created that lacked the α7 helix to analyze the role of α7 helix in PTP1B activity regulation. PTP1B_{1-284 Δ7} was observed to have a size of 2.0 Å based on x-ray crystallography analysis. In one embodiment, PTP1B_{1-284 Δ7} has an amino acid sequence similar to SEQ ID NO: 3. Similar to PTP1B_{Δ7}, PTP1B_{1-284 P185G} and PTP1B_{1-284 P185G} bound to TCS-401 were observed to have a size of 2.0 Å.

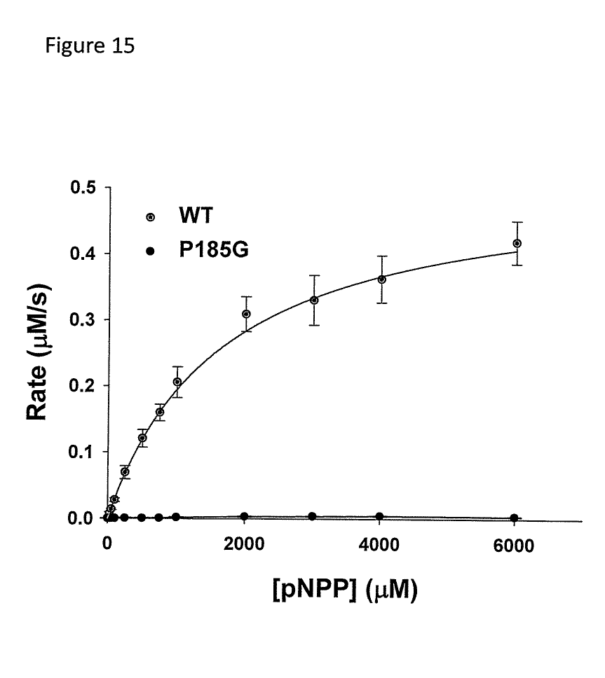

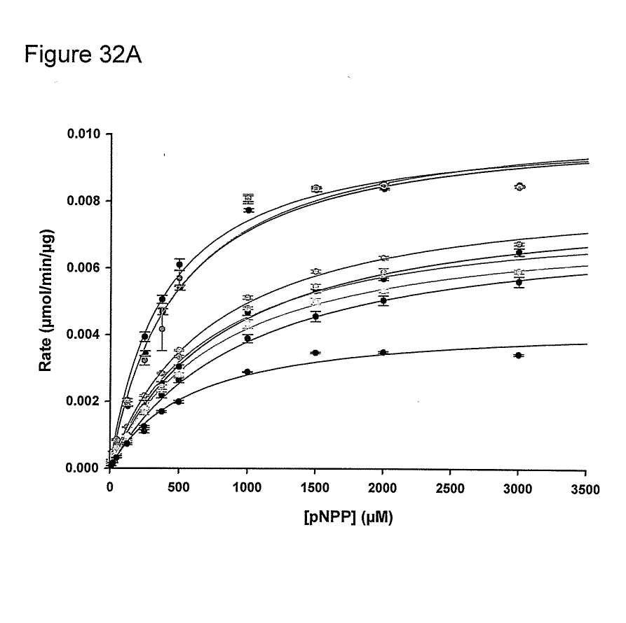

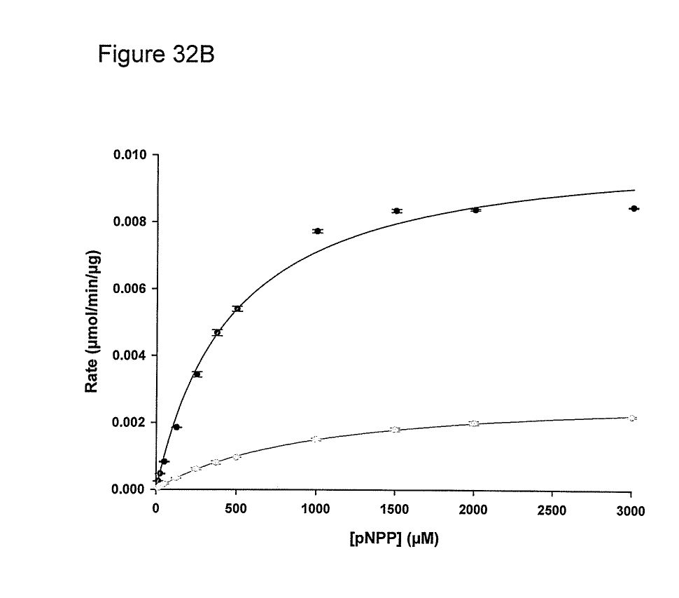

An enzyme assay was used to compare the activity of wild-type PTP1B₁₋₃₀₁ to the activity of PTP1B_{1-284 P185G}. See, FIG. 15. Enzyme assays were performed at pH 7.5 with 0.015 μM PTP1B to compare the activities of wild-type PTP1B₁₋₃₀₁ to mutants with mutations in the α7 helix. See, FIG. 32A. In one embodiment, these point mutations were made in an amino acid sequence substantially similar to SEQ ID NO: 2.

Wild-type PTP1B₁₋₃₀₁ (blue) was observed to have a Kₘ of 0.46±0.06 mM, k_{cat} of 6.2±0.3 s⁻¹, and a melting point (Tₘ) of 55.46° C. Ibid. PTP1B_{D298A} (light blue) was observed to have a Kₘ of 0.48±0.1 mM and a k_{cat} of 6.3±0.4 s⁻¹. Ibid. PTP1B_{K292A} (gray) was observed to have a Kₘ of 0.38±0.05 mM, k_{cat} of 6.1±0.3 s⁻¹. Ibid. PTP1B_{L294A} (pink) was observed to have a Kₘ of 0.73±0.05 mM and a k_{cat} of 5.1±0.1 s⁻¹. Ibid. PTP1B_{G283P} (purple) was observed to have a Kₘ of 0.85±0.07 mM, a k_{cat} of 4.9±0.2 s⁻¹. Ibid. PTP1B_{E297A} (orange) was observed to have a Kₘ of 0.72±0.12 mM and a k_{cat} of 4.6±0.3 s⁻¹. Ibid. PTP1B_{S295A} (light green) was observed to have a Kₘ of 0.80±0.07 mM and a k_{cat} of 4.5±0.1 s⁻¹. Ibid. PTP1B_{W291A} (dark green) was observed to have a K, of 0.99±0.15 mM and a k_{cat} of 4.5±0.3 s⁻¹. Ibid. PTP1B_{S295A/E297A} (dark green) was observed to have a Kₘ of 0.58±0.13 mM and a k_{cat} of 2.6±0.13 s⁻¹. Ibid.

The greater rigidity of the wild-type PTP1B₁₋₃₀₁ was observed to be important for activity. Ibid. PTP1B_{Δ7} was observed to lack the activity exhibited by wild-type PTP1B₁. See, FIG. 32B. Wild-type PTP1B₁₋₃₀₁ (blue) was observed to have a Kₘ of 0.46±0.06 mM, k_{cat} of 6.2±0.3 s⁻¹, and a melting point (Tₘ) of 55.46° C., while PTP1B_{1-284/Δ7} was observed to have a Kₘ of 0.97±0.1 mM, k_{cat} of 1.7±0.07 s⁻¹, and a melting point (Tₘ) of 54.9° C. Ibid.

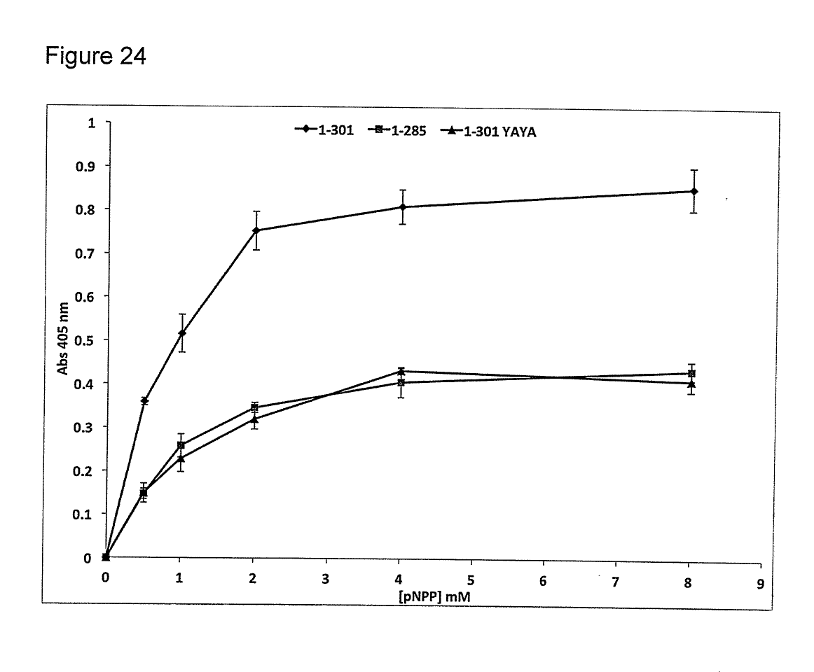

PTP1B_{Δ7} and PTP1B_{YAYA} were observed to have similar phosphatase activity, which was much lower than phosphatase activity of wild-type PTP1B₁₋₃₀₁. See, FIG. 24.

### Example 12

### Analysis of PTP1B_{Δ7} Bound to Active Site Inhibitor TCS-401

Using x-ray crystallography, PTP1B₁₋₂₈₄ bound to TCS-401 was observed to have a size of 1.8 Å.

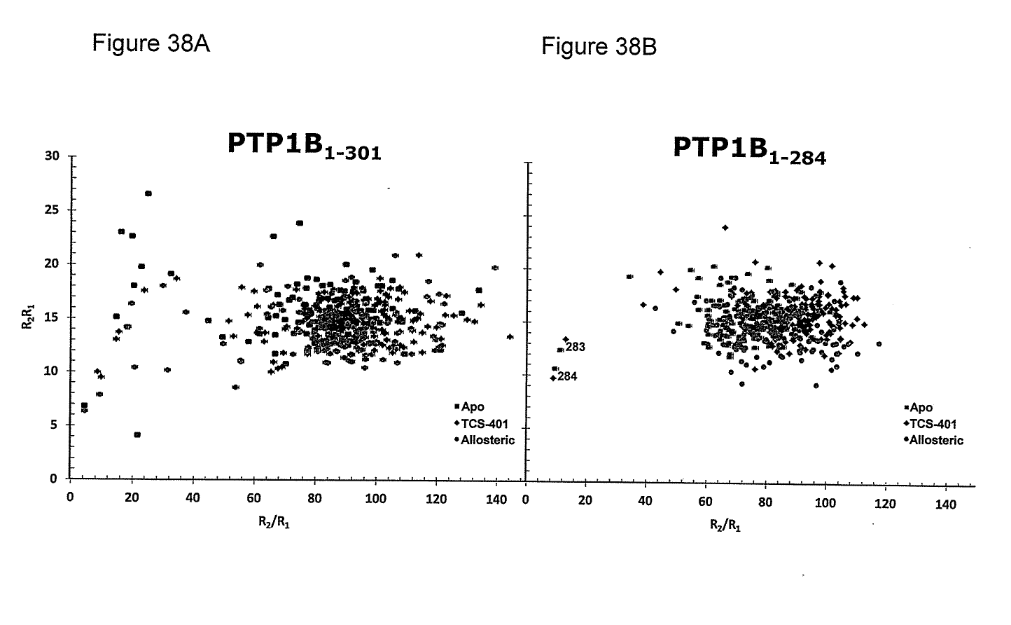

To determine the effect of removing the α7 helix on motion distribution in each enzyme, an overlay of R₂R₁ versus R₂/R₁ values for apo wild-type PTP1B₁₋₃₀₁ (squares), wild-type PTP1B₁₋₃₀₀ bound to TCS-401 (diamonds), and wild-type PTP1B₁₋₃₀₁ bound to an allosteric inhibitor (circles) was compared to an overlay of R₂R₁ versus R₂/R₁ values for apo PTP1B₁₋₂₈₄ (squares), PTP1B₁₋₂₈₄ bound to TCS-401 (diamonds), and PTP1B₁₋₂₈₄ bound to an allosteric inhibitor (circles). See, FIG. 38A and FIG. 38B.

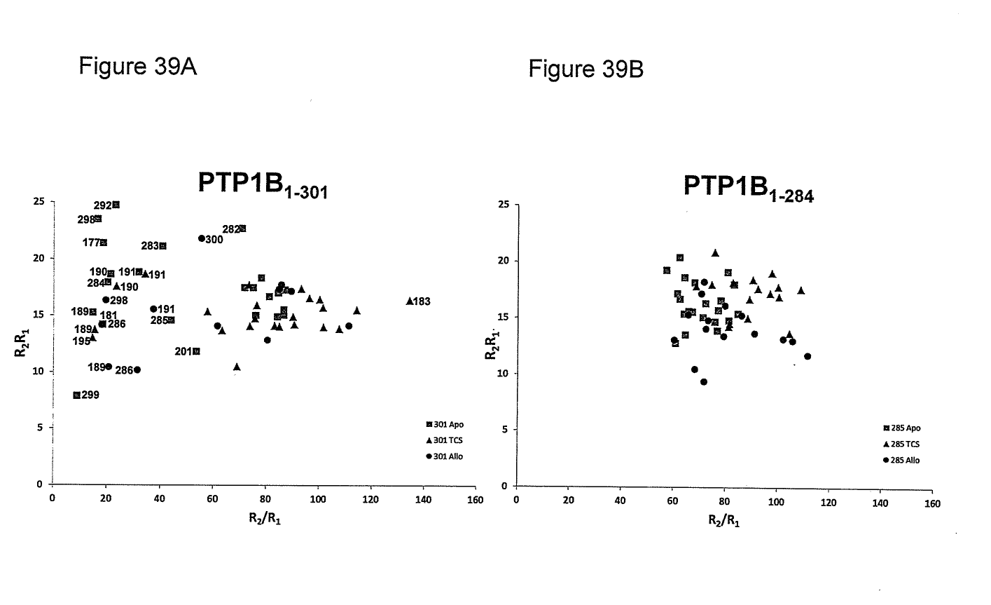

FIG. 39A and FIG. 39B are overlays of the R₂R₁ versus R₂/R₁ for amino acid residues 175-205 (WPD loop and α3 helix) and 275-301 (α6 helix and α7 helices) within wild-type PTP1B₁₋₃₀₁ (squares), wild-type PTP1B₁₋₃₀₁ bound to TCS-401 (triangles), and wild-type PTP1B₁₋₃₀₁ bound to an allosteric inhibitor (circles), PTP1B₁₋₂₈₄ (squares), PTP1B₁₋₂₈₄ bound to TCS-401 (triangles), and PTP1B₁₋₂₈₄ bound to an allosteric inhibitor (circles).

Truncating α7 helix was observed to reduce distribution of motions in mobile regions of PTP1B.

### Example 13

### Analysis of Apo PTP1B_{L192A} and Bound to Active Site Inhibitor TCS-401

X-ray crystallography indicated that PTP1B_{L192A} had a size of 2.0 Å, and PTP1B_{L192A} bound to TCS401 had a size of 2.1 Å.

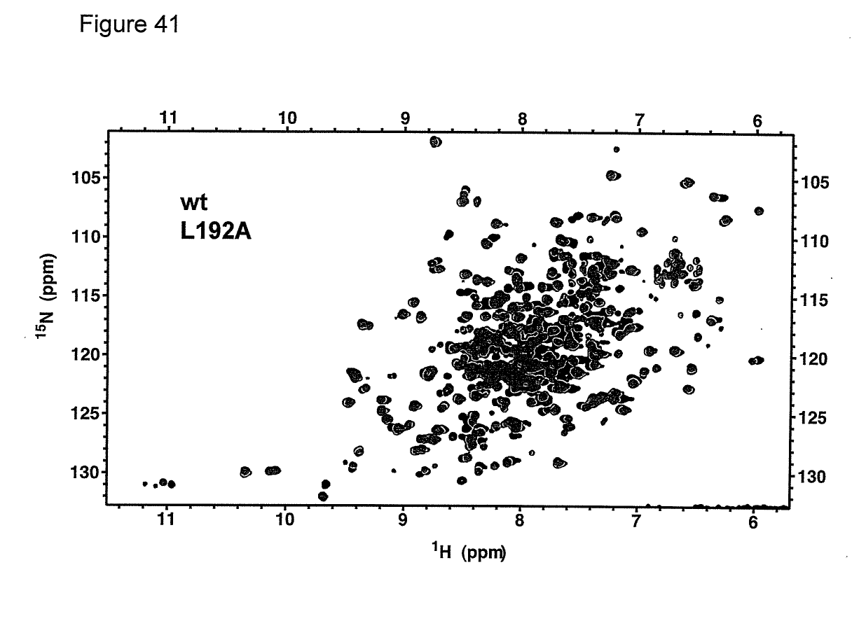

TROSY spectra at 850 MHz for each of wild-type PTP1B_{L192A} (red) and wild-type PTP1B₁₋₃₀₁ (black) are shown in FIG. 41.

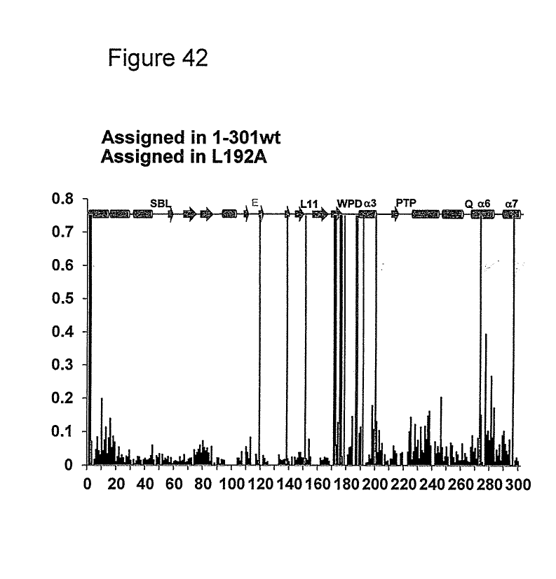

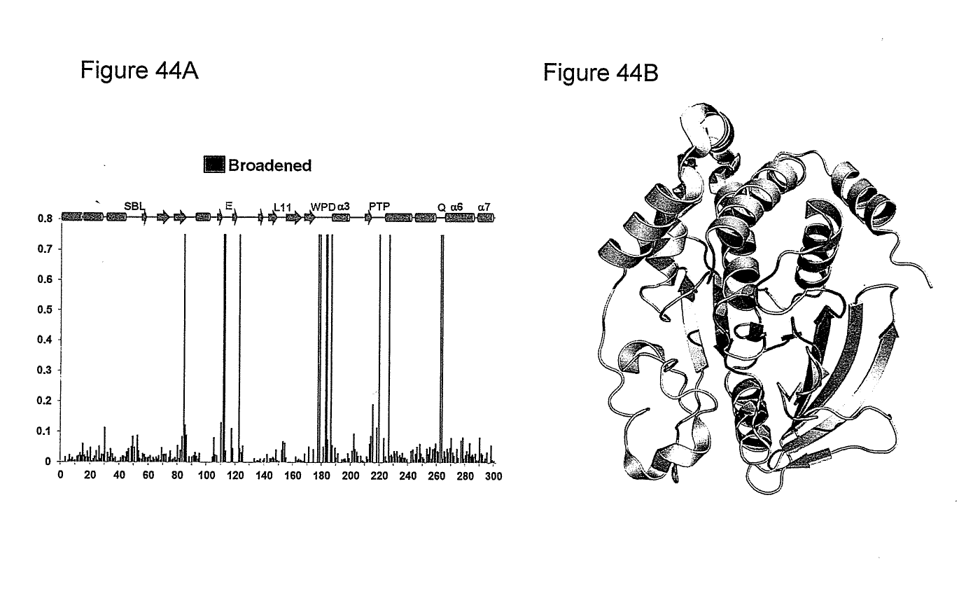

CS observed at 850 MHz indicated changes in dynamics for each PTP1B_{L192A} mutation and PTP1B_{L192A} bound to TCS-401 as compared to wild-type PTP1B. See, FIG. 42. For CS analysis of PTP1B_{L192A} bound to TCS-401, 76% of the backbone was assigned by ¹⁵N, as well as 3 out of 10 in the WPD loop, 9 out of 11 in the α3 helix, and 11 out of 12 in the α7 helix. See, FIG. 44A. The ribbon model of FIG. 44B illustrates these results, and points out in red amino acid residues having a CS greater than the mean plus 2σ

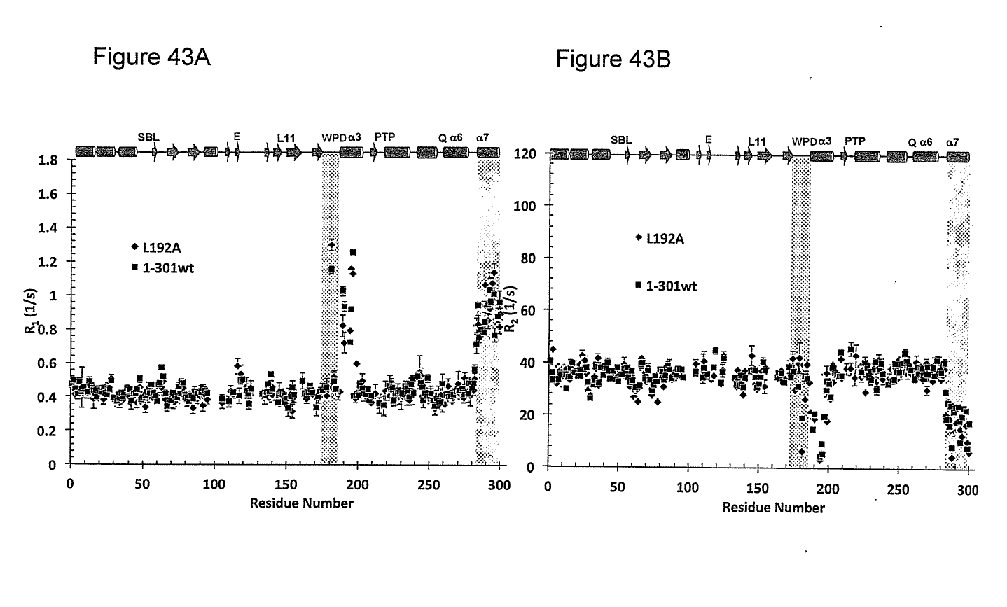

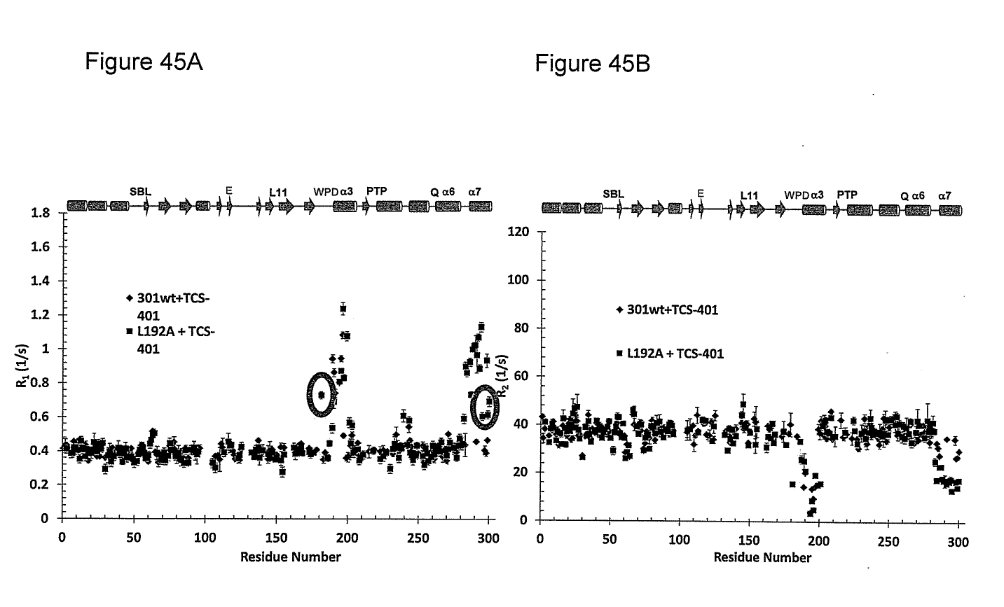

Changes in motions of the domains of wild-type PTP1B₁₋₃₀₁ were compared to the changes in PTP1B_{L192A} by detecting nuclear relaxation time data (R₁ and R₂) at 850 MHz. See, FIG. 43A and FIG. 43B. The effects of binding of an active site inhibitor were measured also by nuclear relaxation time data (R₁ and R₂) of wild-type PTP1B₁₋₃₀₁ bound to TCS-401 and PTP1B_{L192A} bound to TCS-401 at 850 MHz. See, FIG. 45A and FIG. 45B.

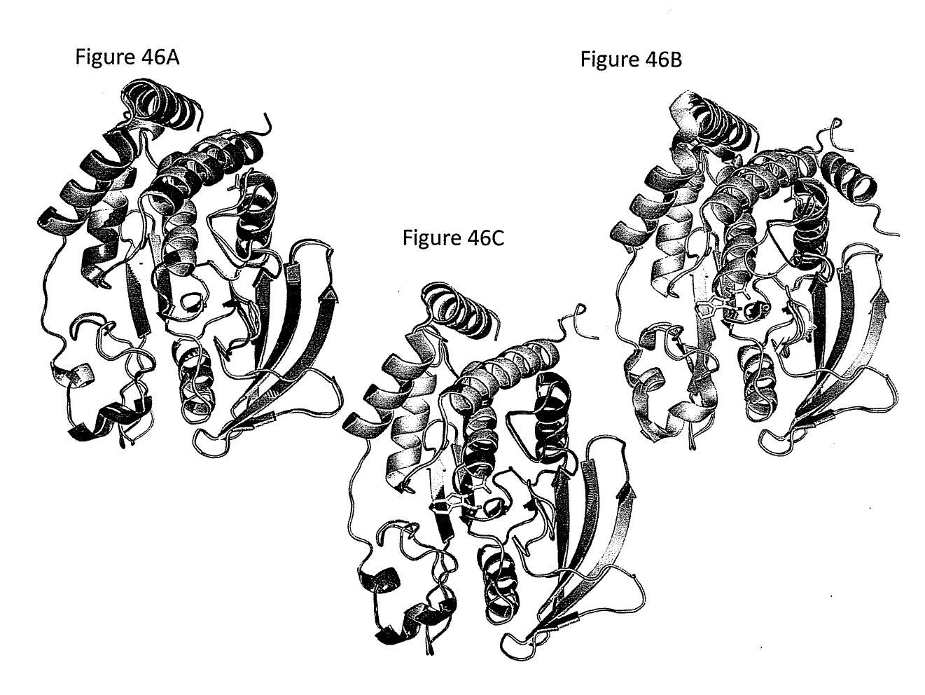

Structural variations were observed among wild-type PTP1B, wild-type PTP1B bound to TCS-401, PTP1B_{L192A}, and PTP1B_{L192A} bound to TCS-401. See, FIG. 46A, FIG. 46B, and FIG. 46C.

Additional mutants were observed using x-ray crystallography to have the following sizes: PTP1B_{H175A} apo (1.9 Å); PTP1B_{H175A} bound to TCS-401 (2.0 Å); PTP1B_{W291A} apo (2.7 Å); PTP1B_{1-284 P185G} bound to TCS-401 (2.0 Å).

### Example 14

### Additional Embodiments

Full sequence specific backbone assignments of wild-type PTP1B, PTP1B_{YAYA}, PTP1B_{N193A}, PTP1B_{L192A}, PTP1B_{T178A} as observed by ¹⁵N-R₁, ¹⁵N-R₂, and hetNOE NMR data are provided herein for the proteins in apo state and during titrations with active site inhibitor TCS-401 and allosteric site inhibitors.

The Examples herein demonstrate the utility of the following amino acid residues of PTP1B: Tyr152, Tyr153, His175, Thr178, Pro185, Phe191, Leu192, Asn193, Trp291, Ser295, and Glu297 to regulate catalytic activity to treat PTP1B related diseases. Amino acid residue His175 was observed to connect the WPD loop with the E loop. Amino acid residue Phe191 was observed to allow movement of amino acid residues Phe191 and Pro185. Leu192 was observed to influence 3D spacing of helix α3 and α6, thus indirectly controlling the catalytic activity of PTP1B. These amino acid residues form the functional spine of PTP1B and modulation of these amino acid residues with pharmaceuticals will allosterically modulate activity of PTP1B.

What is claimed is:

1. A method of treating a subject for a PTP1B related disease, the method comprising the steps of:
administering to a subject a composition that binds to at least one portion of PTP1B protein distal to a catalytic site thereby changing the conformation of enzymatic PTP1B protein;
inhibiting at least one function of PTP1B compared to the PTP1B function prior to the administering; and
decreasing a symptom of the PTP1B related disease, thereby treating the subject for the disease.

2. The method according to claim 1, wherein the portion of the PTP1B protein is at least one of an α7 helix, an α3 helix, an α3 helix, an L11 loop, an E loop, and an WPD loop.

3. The method according to claim 1, wherein the PTP1B has an amino acid sequence selected from the group consisting of: SEQ ID NO: 1, SEQ ID NO: 2, SEQ ID NO: 3, and conservative substitutions thereof.

4. The method according to claim 1, wherein the portion of PTP1B is a functional spine comprising at one amino acid residue selected from the group consisting of: Tyr152, Tyr153, His175, Thr178, Pro185, Phe191, Leu192, Asn193, Cys215, Gly218, Phe280, Val287, Trp291, Lys292, Leu294, Ser295, and Glu297.

5. The method according to claim 1, wherein the changing step further comprises stabilizing the enzyme in an inactive form.

6. The method according to claim 1, wherein the PTP1B related disease is at least one disease selected from the group consisting of cancer, obesity, and diabetes.

7. The method according to claim 1, wherein the changing step further comprising allosterically modulating catalytic activity of PTP1B.

8. The method according to claim 1, wherein during the inhibiting step, a WPD loop remaining in an open conformation.

9. A composition for treating a PTP1B related disease comprising:
a molecule that binds to a portion of human PTP1B protein distal to a catalytic site thereby changing the conformation of PTP1B, wherein at least one function of the PTP protein is inhibited.

10. The composition according to claim 9, wherein the molecule binds to at least one amino acid residue selected from the group consisting of a tyrosine in a L11 loop, a histidine between the L11 loop and a WPD loop, a threonine in the WPD loop, a proline in an α3 helix, a phenylalanine in the α3 helix, a leucine in the α3 helix, an asparagine in the α3 helix, a cysteine in a PTP loop, a glycine in a PTP loop, a phenylalanine in an α6 loop, a valine in the α6 loop, a tryptophan in an α7 helix, a lysine in the α7 helix, a leucine in the α7 helix, a serine in the α7 helix, and a glutamic acid in the α7 helix.

11. The composition according to claim 9, wherein the molecule is a noncompetitive inhibitor.

12. The composition according to claim 9, wherein the molecule is a competitive inhibitor.

13. The composition according to claim 9, wherein the catalytic site is an active site.

14. The composition according to claim 9, wherein the PTP1B protein has an amino acid sequence is at least one selected from the group consisting of SEQ ID NO: 1, SEQ ID NO: 2, SEQ ID NO: 3, and conservative substitutions thereof.

15. The composition according to claim 9, wherein the molecule has an amino acid sequence comprising at least one amino acid sequences selected from the group consisting of SEQ ID NO: 1, SEQ ID NO: 2, SEQ ID NO: 3, and conservative substitutions thereof.

16. The composition according to claim 9, wherein the α7 domain comprises glycine at amino acid position 277, phenylalanine at amino acid position 280, valine at amino acid position 287, lysine at amino acid position 292, leucine at amino acid position 294, and serine at amino acid position 295.

17. A method of identifying a peptide that inhibits PTP1B, the method comprising:
contacting a mixture of purified PTP1B with a peptide library containing at least one peptide having an amino acid sequence selected from the group consisting of: SEQ ID NO: 1, SEQ ID NO: 2, SEQ ID NO: 3, and conservative substitutions thereof;
isolating at least one peptide that bind PTP1B and alter conformation thereby inhibiting PTP1B; and
determining and aligning amino acid sequences of the peptides that bind PTP1B and alter conformation to identify amino acid positions with conserved amino acids.

18. The method according to claim 17, wherein the isolating step further comprising isolating the peptide that restricts movement of amino acid residues Phe191 or Pro185, alters spacing of an α3 helix, or interferes with connection of a WPD loop with an E-loop.

19. The method according to claim 17, wherein the isolating step comprises isolating the peptide that bind PTP1B distal to the active site.

20. The method according to claim 17, wherein the isolating step further comprising isolating peptides that regulate the catalytic cycle of PTP1B.


SEQUENCE LISTING (3 sequences)

SEQ ID NO: 1 - protein, 321 aa, Artificial Sequence
  The sequence was designed and synthesized
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN

SEQ ID NO: 2 - protein, 301 aa, Artificial Sequence
  The sequence was designed and synthesized
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEP

SEQ ID NO: 3 - protein, 284 aa, Artificial Sequence
  The sequence was designed and synthesized
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGD